(19) United States
(12) Patent Application Publication
(10) Pub. No.: US 2009/0209535 A1
(43) Pub. Date: Aug. 20, 2009

(54) 6-ETHER/THIOETHER-PURINES AS TOPOISOMERASE II CATALYTIC INHIBITORS AND THEIR USE IN THERAPY

(75) Inventors: Lars Hollund Jensen, Copenhagen (DK); Maxwell Sehested, Copenhagen (DK)

Correspondence Address: MICHAEL BEST & FRIEDRICH LLP, ONE SOUTH PINCKNEY STREET, P O BOX 1806, MADISON, WI, 53701, US

(73) Assignee: TOPOTARGET A/S, Copenhagen (DK)

(21) Appl. No.: 11/815,782

(22) PCT Filed: Feb. 8, 2006
(86) PCT No.: PCT/IB2006/000377

§ 371 Date: Feb. 6, 2009

(30) Foreign Application Priority Data
Feb. 8, 2005 (GB) 0502573.9

(51) Int. Cl. A61K 31/5377; A61K 31/52; C12N 5/02

(52) U.S. Cl. 514/234.2; 514/263.1; 435/375

(57) ABSTRACT

The present invention relates to certain purines of the following formulae, which act as topoisomerase II catalytic inhibitors: wherein: J is independently: —H or —NR^{N1}R^{N2}; X is independently: —O—, or —S—; Q is independently: a covalent bond, C₁₋₇alkylene, C₂₋₇alkenylene, C₂₋₇alkynylene, C₃₋₇cycloalkylene, C₃₋₇cycloalkenylene, or C₃₋₇cycloalkynylene; T is independently: a group A¹ or a group A²; A¹ is independently: C₆₋₁₄carboaryl, C₅₋₁₄heteroaryl, C₃₋₁₂carbocyclic, or C₃₋₁₂heterocyclic; and is independently unsubstituted or substituted; A² is independently: —H, —CN, —OH, or —O(C═O)—C₁₋₇alkyl; R^{N} is independently —H or a nitrogen ring substituent; R⁸ is independently —H or a ring substituent; either: each of R^{N1} and R^{N2} is independently —H or a nitrogen substituent; or: R^{N1} and R^{N2} taken together with the nitrogen atom to which they are attached form a ring having from 3 to 7 ring atoms; and pharmaceutically acceptable salts, solvates, amides, esters, ethers, N-oxides, chemically protected forms, and prodrugs thereof. These compounds are useful in combination with topoisomerase II poisons, such as anthracyclines and epipodophyllotoxins, in the treatment of proliferative conditions (e.g., cancer). These compounds are also useful in the treatment of tissue damage associated with extravasation of a topoisomerase II poison, such as an anthracycline or an epipodophyllotoxin.

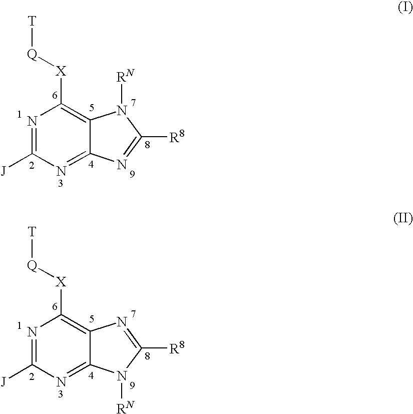

### RELATED APPLICATION

This application is related to: United Kingdom patent application 0502573.9 filed 8 Feb. 2005, the contents of which are incorporated herein by reference in their entirety.

### TECHNICAL FIELD

The present invention relates to topoisomerase II catalytic inhibitors, and their use in therapy. In particular, the present invention relates to certain purines (6-ether/thioether-purines) and derivatives thereof for use in combination with cytostatic agents that act as topoisomerase II poisons, such as anthracyclines and epipodophyllotoxins, in the treatment of proliferative conditions (e.g., cancer). The present invention also relates to use of these compounds in the treatment of tissue damage associated with accidental extravasation of a topoisomerase II poison, such as an anthracycline or an epipodophyllotoxin.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiments.

### Topoisomerase II

Topoisomerase II is an essential nuclear enzyme found in all living cells. The basic activity of this enzyme is to transiently create a double strand break in one DNA molecule through which a second double stranded DNA molecule is transported (see, e.g., Roca and Wang, 1994). During this gating process, topoisomerase II is covalently attached to DNA, and this configuration of topoisomerase II covalently attached to DNA is called the cleavage complex (see, e.g., Wilstermann and Osheroff, 2003). Topoisomerase II participates in various DNA metabolic processes such as transcription, DNA replication, chromosome condensation, and de-condensation, and is essential at the time of chromosome segregation following cell division (see, e.g., Wang, 2002). While lower eukaryotes have only one type II topoisomerase, higher vertebrates have two isoforms, namely α (alpha) and β (beta). Topoisomerase II α is essential for cell proliferation and is expressed only in dividing cells (see, e.g., Wang, 2002). The β isoform is not required for cell proliferation, but knockout mice lacking this isoform die shortly after birth due to defects in their central nervous system (see, e.g., Yang, 2000).

Compared to compounds that target the activity of the mitotic spindle apparatus, topoisomerase II directed drugs are among the most successful clinically applied anti-cancer compounds, and encompass such important classes as: epipodophyllotoxins (exemplified by etoposide), aminoacridines (exemplified by amsacrine), and anthracyclines (exemplified by doxorubicin, daunorubicin and idarubicin) (see, e.g., Larsen et al., 2003). The success of topoisomerase II as an anti-cancer target relates to its essential role in cells, its selective expression in proliferating cells (the α isoform), and its lack of biological redundancy.

Most topoisomerase II-directed compounds currently in clinical use, like the ones mentioned above, work by a rather unusual mechanism. Instead of inhibiting the catalytic activity of topoisomerase II, these compounds increase the levels of covalent cleavage complexes in cells (see, e.g., Wilstermann and Osheroff, 2003). The action of DNA metabolic processes then renders these complexes into permanent double strand breaks, which are highly toxic to cells (see, e.g., Li and Liu, 2001). Topoisomerase II poisons display some level of cancer selectivity due to the fact that malignant cells tend to divide more rapidly than cells in normal tissues and that they have high levels of topoisomerase II α expression. Despite these facts, all topoisomerase II poisons clinically used are toxic to several types of rapidly dividing cells in normal tissues, such as the bone marrow and the gut lining, causing these compounds to have unwanted side effects. One possible way of improving cancer selectivity is to modulate the activity of known topoisomerase II poisons by the use of topoisomerase II catalytic inhibitors (see, e.g., Jensen and Sehested, 1997). Several classes of structurally unrelated compounds, including the anthracycline derivative aclarubicin (see, e.g., Jensen et al., 1990; Nitiss et al., 1997), the conjugated thiobarbituric acid derivate merbarone (see, e.g., Drake et al., 1989), the coumarin drugs novobiocin and cumermycine (see, e.g., Goto and Wang, 1982), the epipodophyllotoxin analog F 11782 (see, e.g., Perrin et al., 2000), fostrecin (see, e.g., Boritzki et al., 1998), chloroquine (see, e.g., Langer et al., 1999; Jensen et al., 1994), maleimide (see, e.g., Jensen et al., 2002), and bisdioxopiperazines such as ICRF-187, ICRF-193, and ICRF-154 (see, e.g., Ishida et al., 1991; Tanabe et al., 1991) have been demonstrated to act as catalytic inhibitors of eukaryotic topoisomerase II. See, for example, the extensive reviews in Andoh and Ishida, 1998, and Larsen et al., 2003.

The bisdioxopiperazine compounds have been shown to antagonize DNA damage and cytotoxicity of the topoisomerase II poisons (see, e.g., Jensen and Sehested, 1997; Hasinoff et al., 1996; Ishida et al., 1996; Sehested et al., 1993; Sehested and Jensen, 1996). That antagonism can be extended to in vitro settings, where ICRF-187 antagonises the effect of etoposide in mice (see, e.g., Holm et al., 1996), thereby allowing etoposide dose-escalation resulting in improved targeting of tumours in the central nervous system. In a similar fashion, aclarubicin has been demonstrated to protect human cells from the action of topoisomerase II poisons (see, e.g., Jensen et al., 1990), an antagonism that has also been extended to an in vivo model (see, e.g., Holm et al., 1994). Finally, chloroquine has been shown to protect human cancer cells from etoposide- and camptothecin-induced DNA breaks and cytotoxicity in a pH-dependent fashion (see, e.g., Sorenson et al., 1997; Jensen et al., 1994) serving as proof of principle that topoisomerase catalytic inhibitors can modulate the activity of topoisomerase poisons by targeting their cytotoxicity to acid environments such those found in solid tumours.

There is a recognized need for more and better treatments for proliferative conditions (e.g., cancer) that offer, for example, one or more the following benefits:

(a) improved activity;
(b) improved efficacy;
(c) improved specificity;
(d) reduced toxicity (e.g., cytotoxicity);
(e) complement the activity of other treatments (e.g., chemotherapeutic agents);
(f) reduced intensity of undesired side-effects;
(g) fewer undesired side-effects;
(h) simpler methods of administration (e.g., route, timing, compliance);
(i) reduction in required dosage amounts;
(j) reduction in required frequency of administration;
(k) increased ease of synthesis, purification, handling, storage, etc.;
(l) reduced cost of synthesis, purification, handling, storage, etc.

Thus, one aim of the present invention is the provision of active compounds that offer one or more of the above benefits.

### SUMMARY OF THE INVENTION

One aspect of the invention pertains to certain active compounds, specifically, certain purines and derivatives thereof as described herein, which act, for example, as topoisomerase II catalytic inhibitors.

Another aspect of the invention pertains to a composition comprising a compound as described herein and a pharmaceutically acceptable carrier or diluent.

Another aspect of the present invention pertains to a compound as described herein for use in a method of treatment of the human or animal body by therapy.

Another aspect of the present invention pertains to a compound as described herein for use in combination with a topoisomerase II poison, such as an anthracycline or an epipodophyllotoxin, in a method of treatment of the human or animal body by therapy.

Another aspect of the present invention pertains to use of a compound, as described herein, in the manufacture of a medicament for use in treatment.

Another aspect of the present invention pertains to use of a compound, as described herein, in the manufacture of a medicament for use in combination with a topoisomerase II poison, such as an anthracycline or an epipodophyllotoxin, in treatment.

Another aspect of the present invention pertains to a method of inhibiting (e.g., catalytically inhibiting) topoisomerase II in a cell, in vitro or in vivo, comprising contacting the cell with an effective amount of a compound, as described herein.

Another aspect of the present invention pertains to a method of treatment comprising administering to a patient in need of treatment a therapeutically effective amount of a compound as described herein, preferably in the form of a pharmaceutical composition.

Another aspect of the present invention pertains to a method of treatment comprising administering to a patient in need of treatment a therapeutically effective amount of a compound as described herein, preferably in the form of a pharmaceutical composition, and a topoisomerase II poison, such as an anthracycline or an epipodophyllotoxin.

Another aspect of the present invention pertains to a method of targeting (e.g., the cytotoxicity of; the antitumour effect of, etc.) a topoisomerase II poison, comprising administering a compound as described herein, in combination with said topoisomerase II poison.

In one embodiment, the targeting is targeting to a solid tumour (e.g., the acid microenvironment of a solid tumour). In one embodiment, the targeting is targeting to the central nervous systems (CNS) (e.g., the brain).

Another aspect of the present invention pertains to a method of permitting increased dosage of a topoisomerase II poison in therapy, comprising administering a compound as described herein, in combination with said topoisomerase II poison.

In one embodiment (e.g., of use in methods of therapy, of use in the manufacture of medicaments, of methods of treatment), the treatment is treatment of a disease or condition that is ameliorated by the catalytic inhibition of topoisomerase II.

In one embodiment, the treatment is prevention or treatment of tissue damage associated with (e.g. accidental) extravasation of a topoisomerase II poison, such as an anthracycline or an epipodophyllotoxin.

In one embodiment (e.g., of use in methods of therapy, of use in the manufacture of medicaments, of methods of treatment), the treatment is treatment of a proliferative condition.

In one embodiment, the treatment is treatment of cancer.

In one embodiment, the treatment is treatment of solid tumour cancer.

In one embodiment, the treatment is treatment of a proliferative condition of the central nervous system (CNS). In one embodiment, the treatment is treatment of a tumour of the central nervous system (CNS). In one embodiment, the treatment is treatment of brain cancer.

In one embodiment, the topoisomerase II poison is an anthracycline or an epipodophyllotoxin.

In one embodiment, the topoisomerase II poison is an anthracycline selected from: doxorubicin, idarubicin, epirubicin, aclarubicin, mitoxantrone, dactinomycin, bleomycin, mitomycin, carubicin, pirarubicin, daunorubicin, daunomycin, 4-iodo-4-deoxy-doxorubicin, N,N-dibenzyl-daunomycin, morpholinodoxorubicin, aclacinomycin, duborimycin, menogaril, nogalamycin, zorubicin, marcellomycin, detorubicin, annamycin, 7-cyanoquinocarcinol, deoxydoxorubicin, valrubicin, GPX-100, MEN-10755, and KRN5500.

In one embodiment, the topoisomerase II poison is an epipodophyllotoxin selected from: etoposide, etoposide phosphate, teniposide, tafluposide, VP-16213, and NK-611.

In one embodiment, the topoisomerase II poison is etoposide.

Another aspect of the present invention pertains to a kit comprising (a) a compound, as described herein, preferably provided as a pharmaceutical composition and in a suitable container and/or with suitable packaging; and (b) instructions for use, for example, written instructions on how to administer the active compound.

In one embodiment, the kit further comprises a topoisomerase II poison, preferably provided as a pharmaceutical composition and in a suitable container and/or with suitable packaging.

As will be appreciated by one of skill in the art, features and preferred embodiments of one aspect of the invention will also pertain to other aspect of the invention.

### BRIEF DESCRIPTION OF THE DRAWINGS

FIG. 1 shows the chemical structures of various purine derivatives discussed herein.

FIG. 2 shows two graphs (panel A and panel B) of topoisomerase II inhibition (CPM) versus drug concentration (μM) for ICRF-187 and NSC 35866, for (A) wild-type human topoisomerase II α, and (B) bisdioxopiperazine resistant Y165S mutant human topoisomerase II α.

FIG. 3 shows two graphs (panel A and panel B): the first is a graph of the absolute rate of hydrolysis of ATP (nM/sec) versus concentration of NSC 35866 (μM), with and without DNA, and the second is relative ATPase activity versus concentration of NSC 35866 (μM), with and without DNA.

FIG. 4 shows nine graphs (panels A through I) of relative ATPase activity versus drug concentration (μM) for a range of drugs.

FIG. 5 show a graph of topoisomerase II inhibition (CPM) versus drug concentration (μM) for several thiopurines.

FIG. 6 shows three graphs (panel A, panel B, panel C) of ΔCPM versus concentration (μM) of drug (A: etoposide, B: NSC 35866, C: NSC 35866 plus etoposide) as determined using an assay for level of topoisomerase II-DNA covalent complexes based on phenol-chloroform extraction.

FIG. 7 shows the results of an assay for retention of salt-stable complexes of human topoisomerase II α on circular DNA attached to magnetic beads via a biotin-streptavidin linkage: Lane 1, no drug; Lane 2, 200 μM ICRF-187; Lane 3, 30 μM NSC 35866; Lane 4, 100 μM NSC 35866; Lane 5, 300 μM NSC 35866; Lane 6, 1000 μM NSC 35866; Lane K, 2 μg human topoisomerase II α.

FIG. 8 shows a graph of relative survival of OC—NYH cells (%) versus concentration of NSC35866 (μM), for treatment with NSC35866 alone, and with both etoposide and NSC35866.

FIG. 9 shows a graph of ¹⁴C retention versus ³H retention, as obtained using an alkaline DNA elution assay for detection of DNA fragmentation, for etoposide, NSC35866, and combinations thereof, at various concentrations.

FIG. 10 shows the results of a band depletion assay, where amounts of topoisomerase II α were visualised by western blotting using a topoisomerase II α specific primary antibody: Lane 1, no drug; Lane 2, 200 μM ICRF-187; Lane 3, 200 μM NSC 35866; Lane 4, 500 μM NSC 35866; Lane 5, 1000 μM NSC 35866.

### DETAILED DESCRIPTION OF THE INVENTION

One aspect of the present invention pertains to compounds which may be described as “6-ether/thioether-purines and analogs thereof”, and their surprising and unexpected activity as topoisomerase II catalytic inhibitors.

### Compounds

One aspect of the present invention pertains to compounds of the following formulae:

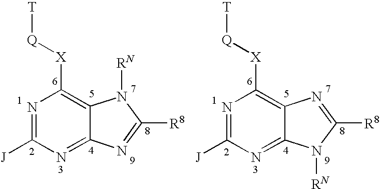

wherein:

J is independently:
—H, or
NR^{N1}R^{N2};

X is independently:
—O—, or
—S—;

Q is independently:
a covalent bond,
C₁₋₇alkylene,
C₂₋₇alkenylene,
C₂₋₇alkynylene,
C₃₋₇cycloalkylene,
C₃₋₇cycloalkenylene, or
C₃₋₇cycloalkynylene;

T is independently:
a group A¹, or
a group A²;

A¹ is independently:
C₆₋₁₄carboaryl,
C₅₋₁₄heteroaryl,
C₃₋₁₂carbocyclic, or
C₃₋₁₂heterocyclic;
and is independently unsubstituted or substituted;

A² is independently:
—H,
—CN,
—OH, or
—O(C═O)—C₁₋₇alkyl;

R^{N} is independently —H or a nitrogen ring substituent;

R⁸ is independently —H or a ring substituent;

either: each of R^{N1} and R^{N2} is independently —H or a nitrogen substituent;

or: R^{N1} and R^{N2} taken together with the nitrogen atom to which they are attached form a ring having from 3 to 7 ring atoms;

and pharmaceutically acceptable salts, solvates, amides, esters, ethers, N-oxides, chemically protected forms, and prodrugs thereof.

### The 7- and 9-Isomers

It should be noted that, when R^{N} is —H, the 7- and 9-isomers exist in dynamic equilibrium in a protic solvent (e.g., in aqueous solution), for example:

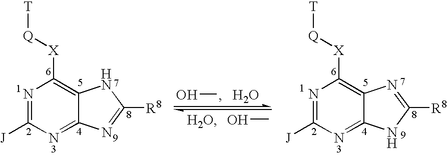

### The 2-Substituent, J

The 2-substituent, J, is independently —H or —NR^{N1}R^{N2}.

In one embodiment, J is independently —H.

In one embodiment, J is independently —NR^{N1}R^{N2}, as in, for example:

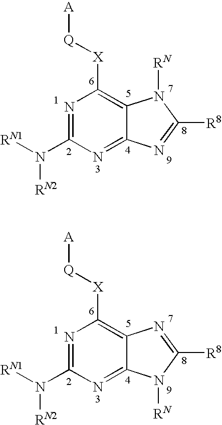

### The Chalcogen Linker, X

The chalcogen linker, X, is independently —O— or —S—.

In one embodiment, X is independently —O—.

In one embodiment, X is independently —S—.

### The Linker, Q

The linker, Q, is independently a covalent bond, C₁₋₇alkylene, C₂₋₇alkenylene, C₂₋₇alkynylene, C₃₋₇cycloalkylene, C₃₋₇cycloalkenylene, or C₃₋₇cycloalkynylene.

In one embodiment, the linker, Q, is a hydrocarbon linker, and is independently C₁₋₇alkylene, C₂₋₇alkenylene, C₂₋₇alkynylene, C₃₋₇cycloalkylene, C₃₋₇cycloalkenylene, or C₃₋₇cycloalkynylene.

In one embodiment, the linker, Q, is independently a covalent bond.

In one embodiment, the linker, Q, is independently as defined herein, but is other than a covalent bond.

The terms “alkylene,” “alkenylene,” etc., as used herein, pertain to bidentate moieties obtained by removing two hydrogen atoms, either both from the same carbon atom, or one from each of two different carbon atoms, of a hydrocarbon compound (a compound consisting of carbon atoms and hydrogen atoms) having from 1 to 20 carbon atoms (unless otherwise specified), which may be aliphatic (i.e., linear or branched) or alicyclic (i.e., cyclic but not aromatic), and which may be saturated, partially unsaturated, or fully unsaturated (but not aromatic).

In one embodiment, Q is independently C₁₋₇alkylene, C₂₋₇alkenylene, or C₂₋₇alkynylene.

In one embodiment, Q is independently C₁₋₄alkylene, C₂₋₄alkenylene, or C₂₋₄alkynylene.

In one embodiment, Q is independently C₁₋₃alkylene, C₂₋₃alkenylene, or C₂₋₃alkynylene.

In one embodiment, Q is independently C₂₋₇alkylene, C₂₋₇alkenylene, or C₂₋₇alkynylene.

In one embodiment, Q is independently C₂₋₄alkylene, C₂₋₄alkenylene, or C₂₋₄alkynylene.

In one embodiment, Q is independently C₂₋₃alkylene, C₂₋₃alkenylene, or C₂₋₃alkynylene.

In one embodiment, Q is independently linear or branched or cyclic.

In one embodiment, Q is independently linear or branched.

In one embodiment, Q is independently linear.

In one embodiment, Q is independently branched.

In one embodiment, Q is independently selected from:

—(CH₂)ₙ— where n is an integer from 1 to 7;

—CH(CH₃)—;

—CH(CH₃)CH₂— and —CH₂CH(CH₃)—;

—CH(CH₃)CH₂CH₂—, —CH₂CH(CH₃)CH₂—, and —CH₂CH₂CH(CH₃)—;

—CH(CH₃)CH₂CH₂CH₂—, —CH₂CH(CH₃)CH₂CH₂—, —CH₂CH₂CH(CH₃)CH₂—, and —CH₂CH₂CH₂CH(CH₃)—;

—CH(CH₃)CH₂CH₂CH₂CH₂—, —CH₂CH(CH₃)CH₂CH₂CH₂—, —CH₂CH₂CH(CH₃)CH₂CH₂—, —CH₂CH₂CH₂CH(CH₃)CH₂—, —CH₂CH₂CH₂CH₂CH(CH₃)—;

—CH(CH₂CH₃)—;

—CH(CH₂CH₃)CH₂— and —CH₂CH(CH₂CH₃)—;

—CH(CH₂CH₃)CH₂CH₂—, —CH₂CH(CH₂CH₃)CH₂—, —CH₂CH₂CH(CH₂CH₃)—;

—CH(CH₂CH₃)CH₂CH₂CH₂—, —CH₂CH(CH₂CH₃)CH₂CH₂—CH₂CH₂CH(CH₂CH₃)CH₂—, and —CH₂CH₂CH₂CH(CH₂CH₃)—;

—CH(CH₂CH₃)CH₂CH₂CH₂CH₂—, —CH₂CH(CH₂CH₃)CH₂CH₂CH₂—, —CH₂CH₂CH(CH₂CH₃)CH₂CH₂—, —CH₂CH₂CH₂CH(CH₂CH₃)CH₂—,

—CH₂CH₂CH₂CH₂CH(CH₂CH₃)—;

—CH═CH—;

—CH═CHCH₂— and —CH₂CH═CH—;

—CH═CHCH₂CH₂—, —CH₂CH═CHCH₂—, and —CH₂CH₂CH═CH₂—;

—CH═CHCH₂CH₂CH₂—, —CH₂CH═CHCH₂CH₂—, —CH₂CH₂CH═CHCH₂—,

—CH₂CH₂CH₂CH═CH—;

—CH═CHCH₂CH₂CH₂CH₂—, —CH₂CH═CHCH₂CH₂CH₂—, —CH₂CH₂CH═CHCH₂CH₂—, —CH₂CH₂CH₂CH═CHCH₂—, —CH₂CH₂CH₂CH₂CH═CH—;

—C(CH₃)═CH— and —CH═C(CH₃)—;

—C(CH₃)═CHCH₂—, —CH═C(CH₃)CH₂—, and —CH═CHCH(CH₃)—;

—CH(CH₃)CH═CH—, —CH₂C(CH₃)═CH—, and —CH₂CH═C(CH₃)—;

—CH═CHCH═CH—;

—CH═CHCH═CHCH₂—, —CH₂CH═CHCH═CH—, and —CH═CHCH₂CH═CH—;

—CH═CHCH═CHCH₂CH₂—, —CH═CHCH₂CH═CHCH₂—, —CH═CHCH₂CH₂CH═CH—, —CH₂CH═CHCH—CHCH₂—, —CH₂CH═CHCH₂CH═CH—, —CH₂CH₂CH═CHCH═CH—;

—C(CH₃)═CHCH═CH—, —CH═C(CH₃)CH═CH—, —CH═CHC(CH₃)═CH—,

—CH═CHCH═C(CH₃)—;

—C≡C—;

—C≡CCH₂—, —CH₂C≡C—; —C≡CCH(CH₃)—, —CH(CH₃)C≡C—;

—C≡CCH₂CH₂—, —CH₂C≡CCH₂—, —CH₂CH₂C≡C—;

—C≡CCH(CH₃)CH₂—, —C≡CCH₂CH(CH₃)—;

—CH(CH₃)C≡CCH₂—, —CH₂C≡CCH(CH₃)—;

—CH(CH₃)CH₂C≡C—, —CH₂CH(CH₃)C≡C—;

—C≡CCH═CH—, —CH═CHC≡C—, —C≡CC≡C—;

—C(CH₃)═CHC≡C—, —CH═C(CH₃)C≡C—, —C≡CC(CH₃)═CH—, —C≡CCH═C(CH₃)—

cyclopentylene and cyclopentenylene;

cyclohexylene, cyclohexenylene, cyclohexadienylene.

In one embodiment, Q is independently selected from:

—CH₂—, —CH₂CH₂—, —CH₂CH₂CH₂—, and —CH₂CH═CH—.

All plausible combinations of the embodiments described above are explicitly disclosed herein, as if each combination was individually and explicitly recited.

In one embodiment, Q is independently selected from —(CH₂)ₙ— where n is an integer from 1 to 7.

In one embodiment, Q is independently selected from —(CH₂)ₙ— where n is an integer from 1 to 4.

In one embodiment, Q is independently selected from —(CH₂)ₙ— where n is an integer from 1 to 3.

In one embodiment, Q is independently —CH₂— or —CH₂CH₂—.

In one embodiment, Q is independently —CH₂—.

In one embodiment, Q is independently —CH₂CH₂—.

### The Nitrogen Ring Substituent

The group R^{N} is independently —H or a nitrogen ring substituent.

In one embodiment, R^{N} is independently —H.

In one embodiment, R^{N} is independently a nitrogen ring substituent.

In one embodiment, the nitrogen ring substituent, if present, is independently selected from:

C₁₋₇alkyl;
C₂₋₇alkenyl;
C₂₋₇alkynyl;
C₃₋₇cycloalkyl;
C₃₋₇cycloalkenyl;
C₃₋₇cycloalkynyl;
C₆₋₂₀carboaryl;
C₅₋₂₀heteroaryl;
C₃₋₂₀heterocyclyl;
C₆₋₂₀carboaryl-C₁₋₇alkyl;
C₅₋₂₀heteroaryl-C₁₋₇alkyl;
C₃₋₂₀heterocyclyl-C₁₋₇alkyl;
and is independently unsubstituted or substituted.

In one embodiment, substitutents on the nitrogen substitutent, if present, are as defined below under the heading “Substituents on the Cyclic Group.”

In one embodiment, the nitrogen ring substituent, if present, is a C₃₋₂₀heterocyclyl group, and is tetrahydrofuranyl, and is independently unsubstituted or substituted (e.g., with one or more groups selected from: —OH, —CH₂OH, —CH₃). Examples of such groups include:

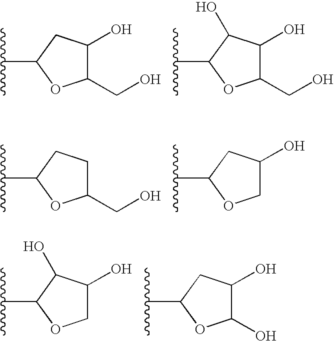

In one embodiment, the nitrogen ring substituent, if present, is a C₃₋₂₀heterocyclyl group, and is ribofuranosyl, e.g., β-ribofuranosyl, D-ribofuranosyl, β-D-ribofuranosyl.

In one embodiment, the nitrogen ring substituent, if present, is a C₃₋₂₀heterocyclyl-C₁₋₇alkyl group, and is morpholino-methyl, piperidino-methyl, or piperazino-methyl, and is independently unsubstituted or substituted (e.g., with one or more groups selected from: —OH, —CH₂OH, —CH₃). Examples of such groups include:

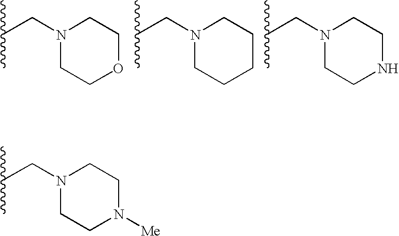

In one embodiment, R^{N} is independently —H or C₁₋₇alkyl, and is independently unsubstituted or substituted.

In one embodiment, R^{N} is independently —H or unsubstituted C₁₋₇alkyl.

In one embodiment, R^{N} is independently —H, -Me, or -Et.

In one embodiment, R^{N} is independently —H or -Me.

In one embodiment, R^{N} is independently —H.

In one embodiment, R^{N} is independently -Me.

In one embodiment, R^{N} is independently selected from:

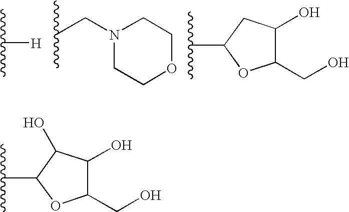

### The Nitrogen Substituents

In one embodiment, the 2-substituent, J, is independently —NR^{N1}R^{N2}.

Either: each of R^{N1} and R^{N2} is independently —H or a nitrogen substituent; or: R^{N1} and R^{N2} taken together with the nitrogen atom to which they are attached form a ring having from 3 to 7 ring atoms.

In one embodiment, each of R^{N1} and R^{N2} is independently —H or a nitrogen substituent.

In one embodiment, each nitrogen substituent is as defined above for nitrogen ring substituents.

In one embodiment, exactly one of R^{N1} and R^{N2} is —H, and the other is a nitrogen substituent.

In one embodiment, neither R^{N1} nor R^{N2} is —H.

In one embodiment, each of R^{N1} and R^{N2} is —H.

In one embodiment, the group —NR^{N1}R^{N2} is independently selected from: —NH₂, —NHMe, —NHEt, —NH(nPr), —NH(iPr), —NH(nBu), —NH(iBu), —NH(sBu), —NH(tBu), —N(Me)₂, —N(Et)₂, —N(nPr)₂, —N(iPr)₂, —N(nBu)₂, —N(iBu)₂, —N(sBu)₂, —N(tBu)₂, —NH(Ph), —N(Ph)₂, —NH(CH₂Ph), —N(CH₂Ph)₂.

In one embodiment, the group —NR^{N1}R^{N2} is independently selected from: —NH₂, —NHMe, —NHEt, —N(Me)₂, —N(Et)₂.

In one embodiment, the group —NR^{N1}R^{N2} is independently —NH₂.

In one embodiment, R^{N1} and R^{N2} taken together with the nitrogen atom to which they are attached form a ring having from 3 to 7 ring atoms.

In one embodiment, the range is from 5 to 7 ring atoms.

In one embodiment, the group —NR^{N1}R^{N2} is independently selected from:

aziridino;
azetidino;
pyrrolidin-N-yl, pyrrolin-N-yl, pyrrol-N-yl;
imidazolidin-N-yl, imidazolin-N-yl, imidazol-N-yl;
pyrazolidin-N-yl, pyrazolin-N-yl, pyrazol-N-yl;
piperidine-N-yl, piperazin-N-yl, pyridin-N-yl;
morpholino;
azepin-N-yl.

### The Terminal Group, T: Cyclic Groups, A¹

In one embodiment, the terminal group, T, is independently a cyclic group, A¹:

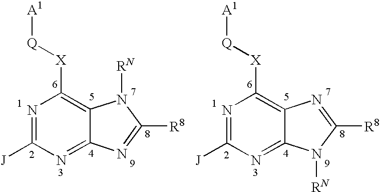

In one embodiment, A¹ is independently:

C₆₋₁₄carboaryl,

C₅₋₁₄heteroaryl,

C₃₋₁₂carbocyclic, or

C₃₋₁₂heterocyclic;

and is independently unsubstituted or substituted.

The term “aryl,” as used herein, pertains to a monovalent moiety obtained by removing a hydrogen atom from an aromatic ring atom of an aromatic compound, which moiety has from 3 to 20 ring atoms (unless otherwise specified). Preferably, each ring has from 5 to 7 ring atoms. The aromatic ring atoms may be all carbon atoms, as in “carboaryl groups” (e.g., phenyl, naphthyl, etc.). Alternatively, the aromatic ring atoms may include one or more heteroatoms (e.g., oxygen, sulfur, nitrogen), as in “heteroaryl groups” (e.g., pyrrolyl, pyridyl, etc.).

The term “carbocyclyl,” as used herein, pertains to a monovalent moiety obtained by removing a hydrogen atom from a non-aromatic ring atom of a carbocyclic compound (a cyclic compound having only carbon ring atoms), which moiety has from 3 to 20 ring atoms (unless otherwise specified). Preferably, each ring has from 3 to 7 ring atoms.

The term “heterocyclyl,” as used herein, pertains to a monovalent moiety obtained by removing a hydrogen atom from a non-aromatic ring atom of a heterocyclic compound (a cyclic compound having at least one ring heteroatom, e.g., oxygen, sulfur, nitrogen), which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms.

Examples of (non-aromatic) monocyclic heterocyclyl groups include those derived from

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted (non-aromatic) monocyclic heterocyclyl groups include saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of carboaryl groups include those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include groups derived from indene (C₉), isoindene (C₉), and fluorene (C₁₃).

Examples of monocyclic heteroaryl groups include those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heterocyclic and heteroaryl groups which comprise fused rings, include those derived from:

C₉heterocyclic and C₉heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀heterocyclic and C₁₀heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₃heterocyclic and C₁₃heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄heterocyclic and C₁₄heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heterocyclic and heteroaryl groups that have a nitrogen ring atom in the form of an —NH— group may be N-substituted, that is, as —NR—. For example, pyrrole may be N-methyl substituted, to give N-methylpyrrole.

Heterocyclic and heteroaryl groups that have a nitrogen ring atom in the form of an —N=group may be substituted in the form of an N-oxide, that is, as —N(→O)═ (also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms. Monocyclic examples of such groups include those derived from:

C₅: cyclopentanone, cyclopentenone, cyclopentadienone;
C₆: cyclohexanone, cyclohexenone, cyclohexadienone;
O₁: furanone (C₅), pyrone (C₆);
N₁: pyrrolidone (pyrrolidinone) (C₅), piperidinone (piperidone) (C₆), piperidinedione (C₆);
N₂: imidazolidone (imidazolidinone) (C₅), pyrazolone (pyrazolinone) (C₅), piperazinone (C₆), piperazinedione (C₆), pyridazinone (C₆), pyrimidinone (C₆) (e.g., cytosine), pyrimidinedione (C₆) (e.g., thymine, uracil), barbituric acid (C₆);
N₁S₁: thiazolone (C₅), isothiazolone (C₅);
N₁O₁: oxazolinone (C₅).

Polycyclic examples of such groups include those derived from:

C₉: indenedione;
C₁₀: tetralone, decalone;
C₁₄: anthrone, phenanthrone;
N₁: oxindole (C₉);
O₁: benzopyrone (e.g., coumarin, isocoumarin, chromone) (C₁₀);
N₁O₁: benzoxazolinone (C₉), benzoxazolinone (C₁₀);
N₂: quinazolinedione (C₁₀);
N₄: purinone (C₉) (e.g., guanine).

Still more examples of cyclic groups which bear one or more oxo (═O) groups on ring carbon atoms include those derived from:

cyclic anhydrides (—C(═O)—O—C(═O)— in a ring), including but not limited to maleic anhydride (C₅), succinic anhydride (C₅), and glutaric anhydride (C₆);
cyclic carbonates (—O—C(═O)—O— in a ring), such as ethylene carbonate (C₅) and 1,2-propylene carbonate (C₅);
imides (—C(═O)—NR—C(═O)— in a ring), including but not limited to, succinimide (C₅), maleimide (C₅), phthalimide, and glutarimide (C₆);
lactones (cyclic esters, —O—C(═O)— in a ring), including, but not limited to, β-propiolactone, γ-butyrolactone, δ-valerolactone (2-piperidone), and ε-caprolactone;
lactams (cyclic amides, —NR—C(═O)— in a ring), including, but not limited to, β-propiolactam (C₄), γ-butyrolactam (2-pyrrolidone) (C₅), δ-valerolactam (C₆), and ε-caprolactam (C₇);
cyclic carbamates (—O—C(═O)—NR— in a ring), such as 2-oxazolidone (C₅);
cyclic ureas (—NR—C(═O)—NR— in a ring), such as 2-imidazolidone (C₅) and pyrimidine-2,4-dione (e.g., thymine, uracil) (C₆).

In one embodiment, A¹ is independently:

C₆₋₁₄carboaryl, or

C₅₋₁₄heteroaryl;

and is independently unsubstituted or substituted.

In one embodiment, A¹ is independently:

C₆₋₁₂carboaryl, or

C₅₋₁₂heteroaryl;

and is independently unsubstituted or substituted.

In one embodiment, A¹ is independently:

C₆₋₁₀carboaryl, or

C₆₋₁₀heteroaryl;

and is independently unsubstituted or substituted.

In one embodiment, A¹ is independently:

monocyclic or bicyclic C₆₋₁₀carboaryl, or

monocyclic or bicyclic C₅₋₁₀heteroaryl;

and is independently unsubstituted or substituted.

In one embodiment, the bicyclic groups are selected from “5-6” fused rings and “6-6” fused rings, e.g., as in benzimidazole and naphthalene, respectively.

In one embodiment, A¹ is independently:

monocyclic C₆carboaryl, or

monocyclic C₅₋₆heteroaryl;

and is independently unsubstituted or substituted.

In one embodiment, the heteroaryl groups have 1, 2, or 3 aromatic ring heteroatoms, e.g., selected from nitrogen and oxygen.

In one embodiment, A¹ is independently derived from one of the following: benzene, naphthylene, pyridine, pyrrole, furan, thiophene, and thiazole; and is independently unsubstituted or substituted.

In one embodiment, A¹ is independently derived from: benzene, naphthylene, pyridine, pyrimidine, imidazole, pyrrole, or benzofurazan; and is independently unsubstituted or substituted.

The phrase “derived from,” as used in this context, pertains to compounds which have the same ring atoms, and in the same orientation/configuration, as the parent heterocycle, and so include, for example, hydrogenated (e.g., partially saturated, fully saturated), carbonyl-substituted, and other substituted derivatives. For example, “pyrrolidone” and “N-methylpyrrole” are both derived from “pyrrole”.

In one embodiment, A¹ is independently: phenyl, naphthyl, pyrididyl, pyrrolyl, furanyl, thienyl, and thiazolyl; and is independently unsubstituted or substituted.

In one embodiment, A¹ is independently: phenyl, naphthyl, pyridyl, pyrimidyl, pyrrolyl, imidazolyl, furanyl, thienyl, thiazoyl, or benzofurazanyl; and is independently unsubstituted or substituted.

In one embodiment, A¹ is independently derived from: benzene, naphthylene, pyridine, or pyrrole; and is independently unsubstituted or substituted.

In one embodiment, A¹ is independently: phenyl, naphthyl, pyridyl, or pyrrolyl; and is independently unsubstituted or substituted.

In one embodiment, A¹ is independently phenyl; and is independently unsubstituted or substituted.

In one embodiment, A¹ is independently a group of the formula:

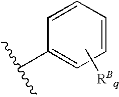

wherein:
q is independently an integer from 0 to 5; and,
each R^{B} is independently a substituent, for example, a monovalent monodentate substituent as defined below under the heading “Substituents on the Cyclic Group.”

The term “monovalent monodentate substituent,” as used herein, pertains to a substituent which has one point of covalent attachment, via a single bond. Examples of such substituents include halo, hydroxy, and alkyl.

In one embodiment, q is independently 0, 1, 2, 3, 4, or 5; or: 1, 2, 3, 4, or 5.

In one embodiment, q is independently 0, 1, 2, 3, or 4; or: 1, 2, 3, or 4.

In one embodiment, q is independently 0, 1, 2, or 3; or: 1, 2, or 3.

In one embodiment, q is independently 0, 1, or 2; or: 1 or 2

In one embodiment, q is independently 0 or 1.

In one embodiment, q is independently 1.

In one embodiment, q is independently 0.

In one embodiment, q is independently 1, and the substituent (e.g., R^{B}) is in a meta or para position.

In one embodiment, A¹ is independently imidazolyl (e.g., 1H-imidazol-5-yl, 1H-imidazol-4-yl); and is independently unsubstituted or substituted (e.g., with one or more substituents selected from -Me, -Et, —NO₂).

In one embodiment, A¹ is independently pyrimidinyl (e.g., pyrimidin-4-yl); and is independently unsubstituted or substituted (e.g., with one or more substituents selected from —Cl, —Br, —SMe, —SEt, —NH₂, —NHMe).

In one embodiment, A¹ is independently benzofurazanyl (e.g., benzofurazan-4-yl, benzofurazan-5-yl); and is independently unsubstituted or substituted (e.g., with one or more substituents selected from —NO₂) (e.g., 7-nitro-benzofurazan-4-yl, 7-nitro-benzofurazan-5-yl).

In one embodiment, A¹ is independently:

C₃₋₁₂carbocyclic (e.g., C₃₋₁₂cycloalkyl, C₃₋₁₂cycloalkenyl), or C₃₋₁₂heterocyclic;

and is independently unsubstituted or substituted.

In one embodiment, A¹ is independently:

C₅₋₁₀carbocyclic (e.g., C₃₋₁₀cycloalkyl, C₃₋₁₀cycloalkenyl), or

C₅₋₁₀heterocyclic;

and is independently unsubstituted or substituted.

In one embodiment, A¹ is independently:

monocyclic or bicyclic C₃₋₁₂carbocyclic (e.g., C₃₋₁₂cycloalkyl, C₃₋₁₂cycloalkenyl), or

monocyclic or bicyclic C₃₋₁₂heterocyclic;

and is independently unsubstituted or substituted.

In one embodiment, the bicyclic groups are selected from “5-6” fused rings and “6-6” fused rings, e.g., as in octahydroindole and decalin, respectively.

In one embodiment, A¹ is independently:

C₅₋₈carbocyclic (e.g., C₅₋₈cycloalkyl, C₅₋₈cycloalkenyl), or

C₅₋₈heterocyclic;

and is independently unsubstituted or substituted.

In one embodiment, A¹ is independently:

monocyclic C₅₋₈carbocyclic (e.g., C₅₋₈cycloalkyl, C₅₋₈cycloalkenyl), or monocyclic C₅₋₈heterocyclic;

and is independently unsubstituted or substituted.

In one embodiment, the heterocyclic groups have 1, 2, or 3 ring heteroatoms, e.g., selected from nitrogen and oxygen.

In one embodiment, A¹ is independently derived from: cyclopentane (e.g., cyclopentyl), cyclohexane (e.g., cyclohexyl), tetrahydrofuran, tetrahydropyran, dioxane, pyrrolidine, piperidine, piperzine; and is independently unsubstituted or substituted (including, e.g., piperidinone, dimethyltetrahydropyran, etc.).

In one embodiment, A¹ is independently: cyclopentyl, cyclohexyl, tetrahydrofuranyl, tetrahydropyranyl, dioxanyl, pyrrolidinyl, piperidinyl, or piperzinyl; and is independently unsubstituted or substituted (including, e.g., piperidinonyl, dimethyltetrahydropyranyl, etc.).

In one embodiment, A¹ is independently cyclohexyl; and is independently unsubstituted or substituted.

In one embodiment, substitutents on the cyclic group, A¹, if present, are as defined below under the heading “Substituents on the Cyclic Group.”

In one embodiment, A¹ is independently selected from those (core groups) exemplified under the heading “Some Preferred Embodiments” and is independently unsubstituted or substituted, for example, with one or more substituents independently selected from those substituents exemplified under the heading “Some Preferred Embodiments.”

In one embodiment, A¹ is independently selected from those groups exemplified under the heading “Some Preferred Embodiments.”

### The Terminal Group, T: Other Groups, A²

In one embodiment, the terminal group, T, is independently a group, A².

In one embodiment, the terminal group, A², is independently:

—H,

—CN,

—OH, or

—O(C═O)—C₁₋₇alkyl.

In one embodiment, the terminal group, A², is independently:

—H,

—CN,

—OH, or

—O(C═O)—C₁₋₇alkyl;

with the proviso that Q is not a covalent bond.

In one embodiment, A² is independently —H, with the proviso that Q is not a covalent bond.

In one embodiment, A² is independently —CN, with the proviso that Q is not a covalent bond.

In one embodiment, A² is independently —OH or —O(C═O)—C₁₋₇alkyl, with the proviso that Q is not a covalent bond.

In one embodiment, A² is independently —OH or —O(C═O)Me, with the proviso that Q is not a covalent bond.

### Substituents on the Cyclic Group

The cyclic group, A¹, is independently unsubstituted or substituted.

In one embodiment, A¹, is independently unsubstituted.

In one embodiment, A¹, is independently substituted.

The term “substituted,” as used herein, pertains to a parent group that bears one or more substituents. The term “substituent” is used herein in the conventional sense and refers to a chemical moiety that is covalently attached to, appended to, or if appropriate, fused to, a parent group. A wide variety of substituents are well known, and methods for their formation and introduction into a variety of parent groups are also well known.

In one embodiment, substituents on the cyclic group A¹ (e.g., R^{B}), if present, are independently selected from:

(1) carboxylic acid; (2) ester; (3) amido or thioamido; (4) acyl; (5) halo; (6) cyano; (7) nitro; (8) hydroxy; (9) ether; (10) thiol; (11) thioether; (12) acyloxy; (13) carbamate; (14) amino; (15) acylamino orthioacylamino; (16) aminoacylamino or aminothioacylamino; (17) sulfonamino; (18) sulfonyl; (19) sulfonate; (20) sulfonamido; (21) oxo; (22) imino; (23) hydroxyimino; (24) C₅₋₂₀aryl-C₁₋₇alkyl; (25) C₅₋₂₀aryl; (26) C₃₋₂₀heterocyclyl; (27) C₁₋₇alkyl; (28) bi-dentate di-oxy groups.

Note that in one embodiment, A¹ is substituted at two positions by a (28) bi-dentate di-oxy group (—O—R—O—), for example, an oxy-C₁₋₃alkyl-oxy group, wherein the C₁₋₃alkyl is unsubstituted or substituted, for example, with halogen, for example fluorine. Examples of such bi-dentate di-oxy groups include —O—CH₂—O—, —O—CH₂—CH₂—O—, —O—CH₂—CH₂—CH₂—O—, —O—CF₂—O—, and —O—CF₂—CF₂—O—. In such cases, A¹ is also optionally substituted by one or more other substituents as described herein.

In one embodiment, the substituents on A¹ (e.g., R^{B}) are independently selected from the following:
(1) —C(═O)OH;
(2) —C(═O)OR¹, wherein R¹ is independently as defined in (24), (25), (26) or (27);
(3) —C(═O)NR²R³ or —C(═S)NR²R³, wherein each of R² and R³ is independently —H; or as defined in (24), (25), (26) or (27); or R² and R³ taken together with the nitrogen atom to which they are attached form a ring having from 3 to 7 ring atoms;
(4) —C(═O)R⁴, wherein R⁴ is independently —H, or as defined in (24), (25), (26) or (27);
(5) —F, —Cl, —Br, —I;
(6) —CN;
(7) —NO₂;
(8) —OH;
(9) —OR⁵, wherein R⁵ is independently as defined in (24), (25), (26) or (27);
(10) —SH;
(11) —SR⁶, wherein R⁶ is independently as defined in (24), (25), (26) or (27);
(12) —OC(═O)R⁷, wherein R⁷ is independently as defined in (24), (25), (26) or (27);
(13) —OC(═O)NR⁸R⁹, wherein each of R⁸ and R⁹ is independently —H; or as defined in (24), (25), (26) or (27); or R⁸ and R⁹ taken together with the nitrogen atom to which they are attached form a ring having from 3 to 7 ring atoms;
(14) —NR¹⁰R¹¹, wherein each of R¹⁰ and R¹¹ is independently —H; or as defined in (24), (25), (26) or (27); or R¹⁰ and R¹¹ taken together with the nitrogen atom to which they are attached form a ring having from 3 to 7 ring atoms;
(15) —NR¹²C(═O)R¹³ or —NR¹²C(═S)R¹³, wherein R¹² is independently —H; or as defined in (24), (25), (26) or (27); and R¹³ is independently —H, or as defined in (24), (25), (26) or (27);
(16) —NR¹⁴C(═O)NR¹⁵R¹⁶ or —NR¹⁴C(═S)NR¹⁵R¹⁶, wherein R¹⁴ is independently —H; or as defined in (24), (25), (26) or (27); and each of R¹⁵ and R¹⁶ is independently —H; or as defined in (24), (25), (26) or (27); or R¹⁵ and R¹⁶ taken together with the nitrogen atom to which they are attached form a ring having from 3 to 7 ring atoms;
(17) —NR¹⁷SO₂R¹⁸, wherein R¹⁷ is independently —H; or as defined in (24), (25), (26) or (27); and R¹⁸ is independently —H, or as defined in (24), (25), (26) or (27);
(18) —SO₂R¹⁹, wherein R¹⁹ is independently as defined in (24), (25), (26) or (27);
(19) —OSO₂R²⁰ and wherein R²⁰ is independently as defined in (24), (25), (26) or (27);
(20) —SO₂NR²¹R²², wherein each of R²¹ and R²² is independently —H; or as defined in (24), (25), (26) or (27); or R²¹ and R²² taken together with the nitrogen atom to which they are attached form a ring having from 3 to 7 ring atoms;
(21) ═O;
(22) ═NR²³, wherein R²³ is independently —H; or as defined in (24), (25), (26) or (27);
(23) ═NOR²⁴, wherein R²⁴ is independently —H; or as defined in (24), (25), (26) or (27);
(24) C₅₋₂₀aryl-C₁₋₇alkyl, for example, wherein C₅₋₂₀aryl is as defined in (25); unsubstituted or substituted, e.g., with one or more groups as defined in (1) to (28);
(25) C₅₋₂₀aryl, including C₅-C₂₀carboaryl and C₅₋₂₀heteroaryl; unsubstituted or substituted, e.g., with one or more groups as defined in (1) to (28);
(26) C₃₋₂₀heterocyclyl; unsubstituted or substituted, e.g., with one or more groups as defined in (1) to (28);
(27) C₁₋₇alkyl, C₂₋₇alkenyl, C₂₋₇alkynyl, C₃₋₇cycloalkyl, C₃₋₇cycloalkenyl, C₃₋₇cycloalkynyl, unsubstituted or substituted, e.g., with one or more groups as defined in (1) to (26) and
(28) —O—R²⁵—, wherein R²⁵ is independently saturated C₁₋₃alkyl, and is independently unsubstituted or substituted with one or more (e.g., 1, 2, 3, 4) substituents as defined in (5).

Some examples of (27) include the following:

halo-C₁₋₇alkyl;

amino-C₁₋₇alkyl (e.g., —(CH₂)_{w}-amino, w is 1, 2, 3, or 4);

amido-C₁₋₇alkyl (e.g., —(CH₂)_{w}-amido, w is 1, 2, 3, or 4);

acylamido-C₁₋₇alkyl (e.g., —(CH₂)_{w}-acylamido, w is 1, 2, 3, or 4);

carboxy-C₁₋₇alkyl (e.g., —(CH₂)_{w}—COOH, w is 1, 2, 3, or 4);

acyl-C₁₋₇alkyl (e.g., —(CH₂)ₙ-acyl, w is 1, 2, 3, or 4);

hydroxy-C₁₋₇alkyl (e.g., —(CH₂)_{w}—OH, w is 1, 2, 3, or 4);

C₁₋₇alkoxy-C₁₋₇alkyl (e.g., —(CH₂)_{w}—O—C₁₋₇alkyl, w is 1, 2, 3, or 4);

In one embodiment, the substituents on A¹ (e.g., R^{B}) are independently selected from the following:
(1) —C(═O)OH;
(2) —C(═O)OMe, —C(═O)OEt, —C(═O)O(iPr), —C(═O)O(tBu); —C(═O)O(cPr);
   —C(═O)OCH₂CH₂OH, —C(═O)OCH₂CH₂OMe, —C(═O)OCH₂CH₂OEt;
   —C(═O)OPh, —C(═O)OCH₂Ph;
(3) —(C═O)NH₂, —(C═O)NMe₂, —(C═O)NEt₂, —(C═O)N(iPr)₂, —(C═O)N(CH₂CH₂OH)₂;
   —(C═O)-morpholino, —(C═O)NHPh, —(C═O)NHCH₂Ph;
(4) —C(═O)H, —(C═O)Me, —(C═O)Et, —(C═O)(tBu), —(C═O)-cHex, —(C═O)Ph; —(C═O)CH₂Ph;
(5) —F, —Cl, —Br, —I;
(6) —CN;
(7) —NO₂;
(8) —OH;
(9) —OMe, —OEt, —O(iPr), —O(tBu), —OPh, —OCH₂Ph;
   —OCF₃, —OCH₂CF₃;
   —OCH₂CH₂OH, —OCH₂CH₂OMe, —OCH₂CH₂OEt;
   —OCH₂CH₂NH₂, —OCH₂CH₂NMe₂, —OCH₂CH₂N(iPr)₂;
   —OPh-Me, —OPh-OH, —OPh-OMe, —OPh-F, —OPh-Cl, —OPh-Br, —OPh-I;
(10) —SH;
(11) —SMe, —SEt, —SPh, —SCH₂Ph;
(12) —OC(═O)Me, —OC(═O)Et, —OC(═O)(iPr), —OC(═O)(tBu); —OC(═O)(cPr);
   —OC(═O)CH₂CH₂OH, —OC(═O)CH₂CH₂OMe, —OC(═O)CH₂CH₂OEt;
   —OC(═O)Ph, —OC(═O)CH₂Ph;
(13) —OC(═O)NH₂, —OC(═O)NHMe, —OC(═O)NMe₂, —OC(═O)NHEt, —OC(═O)NEt₂, —OC(═O)NHPh, —OC(═O)NCH₂Ph;
(14) —NH₂, —NHMe, —NHEt, —NH(iPr), —NMe₂, —NEt₂, —N(iPr)₂, —N(CH₂CH₂OH)₂;
   —NHPh, —NHCH₂Ph; piperidino, piperazino, morpholino;
(15) —NH(C═O)Me, —NH(C═O) Et, —NH(C═O)ₙPr, —NH(C═O)Ph, —NHC(═O)CH₂Ph;
   —NMe(C═O)Me, —NMe(C═O)Et, —NMe(C═O)Ph, —NMeC(═O)CH₂Ph;
(16) —NH(C═O)NH₂, —NH(C═O)NHMe, —NH(C═O)NHEt, —NH(C═O)NPh, —NH(C═O)NHCH₂Ph; —NH(C═S)NH₂, —NH(C═S)NHMe, —NH(C═S)NHEt, —NH(C═S)NPh, —NH(C═S)NHCH₂Ph;
(17) —NHSO₂Me, —NHSO₂Et, —NHSO₂Ph, —NHSO₂PhMe, —NHSO₂CH₂Ph;
   —NMeSO₂Me, —NMeSO₂Et, —NMeSO₂Ph, —NMeSO₂PhMe, —NMeSO₂CH₂Ph;
(18) —SO₂Me, —SO₂CF₃, —SO₂Et, —SO₂Ph, —SO₂PhMe, —SO₂CH₂Ph;
(19) —OSO₂Me, —OSO₂CF₃, —OSO₂Et, —OSO₂Ph, —OSO₂PhMe, —OSO₂CH₂Ph;
(20) —SO₂NH₂, —SO₂NHMe, —SO₂NHEt, —SO₂NMe₂, —SO₂NEt₂, —SO₂-morpholino, —SO₂NHPh, —SO₂NHCH₂Ph;
(21) ═O;
(22) ═NH, ═NMe; ═NEt;
(23) ═NOH, ═NOMe, ═NOEt, ═NO(nPr), ═NO(iPr), ═NO(cPr), ═NO(CH₂-cPr);
(24) —CH₂Ph, —CH₂Ph-Me, —CH₂Ph-OH, —CH₂Ph-F, —CH₂Ph-Cl;
(25) -Ph, -Ph-Me, -Ph-OH, -Ph-OMe, -Ph-NH₂, -Ph-F, -Ph-Cl, -Ph-Br, -Ph-I;
   pyridyl, pyrazinyl, pyrimidinyl, pyridazinyl, furanyl, thiophenyl, pyrrolyl, imidazolyl, pyrazolyl, oxazolyl, thiazolyl, thiadiazolyl;
(26) pyrrolidinyl, imidazolidinyl, pyrazolidinyl, piperidinyl, piperazinyl, azepinyl, tetrahydrofuranyl, tetrahydropyranyl, morpholinyl, azetidinyl;
(27) -Me, -Et, -nPr, -iPr, -nBu, -iBu, -sBu, -tBu, -nPe;
   -cPr, -cHex; —CH═CH₂, —CH₂—CH═CH₂;
   —CF₃, —CHF₂, —CH₂F, —CCl₃, —CBr₃, —CH₂CH₂F, —CH₂CHF₂, and —CH₂CF₃;
   —CH₂OH, —CH₂OMe, —CH₂OEt, —CH₂NH₂, —CH₂NMe₂;
   —CH₂CH₂OH, —CH₂CH₂OMe, —CH₂CH₂OEt, —CH₂CH₂CH₂NH₂, —CH₂CH₂NMe₂;
(28) —O—CH₂—O—, —O—CH₂—CH₂—O—, —O—CH₂—CH₂—CH₂—O—, —O—CF₂—O—, and —O—CF₂—CF₂—O—.

In one embodiment, the substituents on A¹ (e.g., R^{B}) are independently selected from substituents as defined above for: (1), (2), (3), (5), (7), (8), (9), (11), (14), (20), (25), and (27).

In one embodiment, the substituents on A¹ (e.g., R^{B}) are independently selected from substituents as defined above for: (1), (3), (5), (7), (8), (9), (14), (20), (25), and (27).

In one embodiment, the substituents on A¹ (e.g., R^{B}) are independently selected from substituents as defined above for: (2), (5), (7), (8), (9), (11), (14), and (27).

In one embodiment, the substituents on A¹ (e.g., R^{B}) are independently selected from substituents as defined above for: (5), (7), (8), (9), and (27).

In one embodiment, the substituents on A¹ (e.g., R^{B}) are independently selected from:

(2) —C(═O)OMe, —C(═O)OEt;

(5) —F, —Cl, —Br, —I;

(7) —NO₂;

(8) —OH;

(9) —OMe, —OEt;

(11) —SMe, —SEt;

(12) —OC(═O)Me, —OC(═O)Et;

(14) —NH₂, —NHMe, —NHEt, —NMe₂, —NEt₂;

(27) -Me, and -Et.

Unless otherwise specified, included in the above are the well known ionic, salt, and solvate forms of these substituents. For example, a reference to carboxylic acid (—COOH) also includes the anionic (carboxylate) form (—COO⁻), a salt or a solvate thereof. Similarly, a reference to an amino group includes the protonated form (—N+HR¹R²), a salt or a solvate of the amino group, for example, a hydrochloride salt. Similarly, a reference to a hydroxyl group also includes the anionic form (—O⁻), a salt or a solvate thereof.

### The Ring Substituent, R⁸

The group R⁸ is independently —H or a ring substituent.

In one embodiment, R⁸ is independently —H.

In one embodiment, R⁸ is independently a ring substituent.

In one embodiment, the ring substituent, if present, is selected from the monovalent monodentate substituents defined above under the heading “Substituents on the Cyclic Group.” (That is, those groups excluding: (21) oxo; (22) imino; (23) hydroxyimino; and (28) bi-dentate di-oxy groups.)

### Combinations

All plausible combinations of the embodiments described above are explicitly disclosed herein, as if each combination was individually and explicitly recited.

Examples of some preferred combinations include the following

(1) in one embodiment: X is —O— or —S—; Q is a covalent bond, —CH₂—, or —CH₂CH₂—; J is —H or —NH₂; and R⁸ is —H.

(2) in one embodiment: X is —O— or —S—; Q is a covalent bond; J is —H or —NH₂; and R⁸ is —H.

(3) in one embodiment: X is —O— or —S—; Q is —CH₂— or —CH₂CH₂—; J is —H or —NH₂; and R⁸ is —H.

(4) in one embodiment: X is —O— or —S—; Q is a covalent bond, —CH₂—, or —CH₂CH₂—; J is —NH₂; and R⁸ is —H.

(5) in one embodiment: X is —O— or —S—; Q is a covalent bond; J is —NH₂; and R⁸ is —H.

(6) in one embodiment: X is —O— or —S—; Q is —CH₂— or —CH₂CH₂—; J is —NH₂; and R⁸ is —H.

(7) in one embodiment: X is —O— or —S—; Q is a covalent bond, —CH₂—, or —CH₂CH₂—; J is —H; and R⁸ is —H.

(8) in one embodiment: X is —O— or —S—; Q is a covalent bond; J is —H; and R⁸ is —H.

(9) in one embodiment: X is —O— or —S—; Q is —CH₂— or —CH₂CH₂—; J is —H; and R⁸ is —H.

(10) in one embodiment: X is —O— or —S—; Q is a covalent bond, —CH₂—, or —CH₂CH₂—; J is —H or —NH₂; R⁸ is —H; and R^{N} is —H.

(11) in one embodiment: X is —O— or —S—; Q is a covalent bond; J is —H or —NH₂; R⁸ is —H; and R^{N} is —H.

(12) in one embodiment: X is —O— or —S—; Q is —CH₂— or —CH₂CH₂—; J is —H or —NH₂; R⁸ is —H; and R^{N} is —H.

(13) in one embodiment: X is —O— or —S—; Q is a covalent bond, —CH₂—, or —CH₂CH₂—; J is —NH₂; R⁸ is —H; and R^{N} is —H.

(14) in one embodiment: X is —O— or —S—; Q is a covalent bond; J is —NH₂; R⁸ is —H; and R^{N} is —H.

(15) in one embodiment: X is —O— or —S—; Q is —CH₂— or —CH₂CH₂—; J is —NH₂; R⁸ is —H; and R^{N} is —H.

(16) in one embodiment: X is —O— or —S—; Q is a covalent bond, —CH₂—, or —CH₂CH₂—; J is —H; R⁸ is —H; and R^{N} is —H.

(17) in one embodiment: X is —O— or —S—; Q is a covalent bond; J is —H; R⁸ is —H; and R^{N} is —H.

(18) in one embodiment: X is —O— or —S—; Q is —CH₂— or —CH₂CH₂—; J is —H; R⁸ is —H; and R^{N} is —H.

### SOME PREFERRED EMBODIMENTS

Some preferred examples of the compounds include the following:

| | | |
|---|---|---|
| 1. | 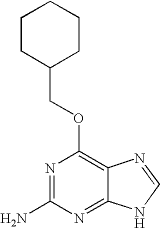 | NU2058 |
| 2. | 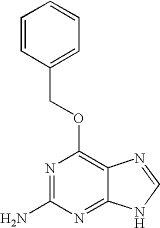 | O⁶-benzylguanine |
| 3. | 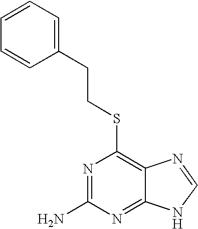 | NSC35866 |
| 4. | 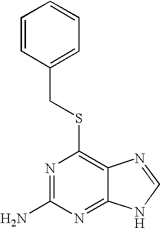 | NSC15747 |
| 5. | 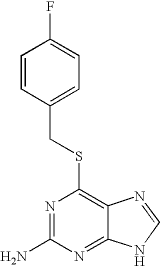 | NSC35865 |
| 6. | 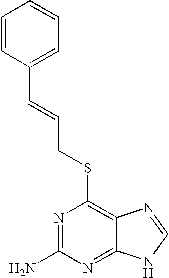 | NSC36824 |
| 7. | 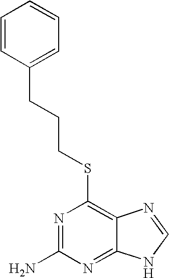 | NSC39328 |
| 8. | 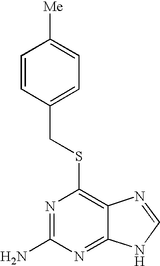 | NSC46384 |

Some additional preferred examples of the compounds include the following:

| | | |
|---|---|---|
| 9. | 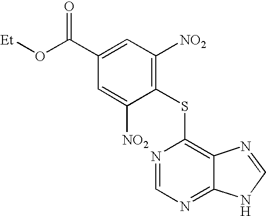 | NSC244708 |
| 10. | 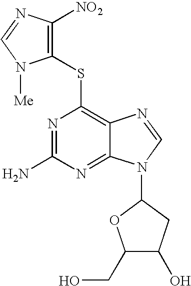 | NSC172614 |
| 11. | 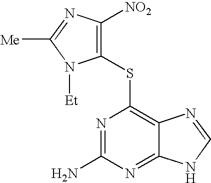 | NSC42375 |
| 12. | 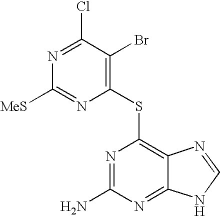 | NSC52383 |
| 13. | 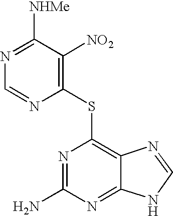 | NSC38732 |
| 14. | 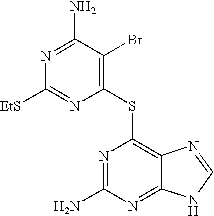 | NSC52388 |
| 15. | 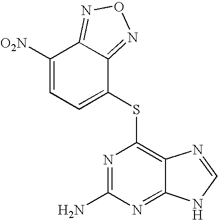 | NSC348401 |
| 16. | 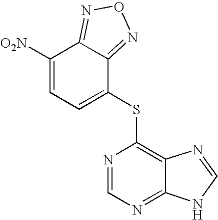 | NSC348402 |
| 17. | 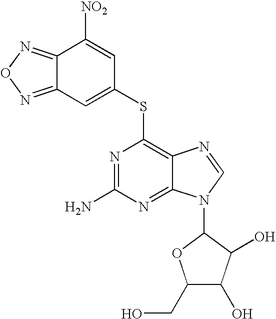 | NSC348400 |

Some additional preferred examples of the compounds include the following:

| | | |
|---|---|---|
| 18. | 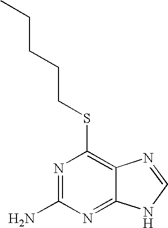 | NSC35862 |
| 19. | 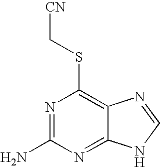 | NSC39331 |
| 20. | 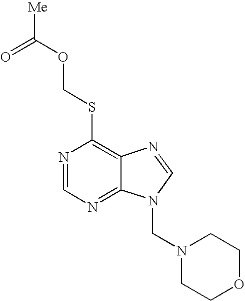 | NSC647471 |

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

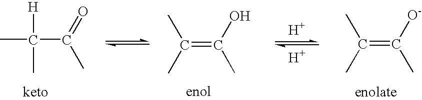

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate forms thereof.

### Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also includes prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several of the active compounds described herein may be obtained from commercial sources, or prepared using well known methods. These and/or other well known methods may be modified and/or adapted in known ways in order to facilitate the synthesis of additional compounds as described herein.

### Uses

Many well known topoisomerase II poisons, including anthracyclines and epipodophyllotoxins, are used in the treatment of proliferative conditions, such as cancer. Without wishing to be bound by any particular theory, it is believed that the compounds described herein (i.e., certain purines and derivatives thereof) act as topoisomerase II catalytic inhibitors. As such, these catalytic inhibitors counter the effects of the poisons. When combined with a partitioning effect, this countering effect may be used to as a means of targeting the effect of the topoisomerase II poison, and thereby provide substantial improvement over treatment with the poison alone, for example, by allowing use of an increased dose of the topoisomerase II poison.

The partitioning effect may arise from the physical, chemical, and/or biological properties of the catalytic inhibitor and/or the poison. For example, the well known topoisomerase II poison etoposide (VP-16) is used in the treatment of proliferative conditions of the central nervous system (CNS) (e.g., brain tumours). The drug is administered systemically and crosses the brain-blood barrier in order to treat the brain tumour. However, the drug also circulates elsewhere in the body, with undesired deleterious effects. By also administering a topoisomerase II catalytic inhibitor which does not (or does not substantially) cross the brain-blood barrier, those undesired deleterious effects can be reduced or eliminated, while not (or not substantially) affecting the desired antitumour effect in the brain. In this way, the topoisomerase II catalytic inhibitor can be used as means of targeting the antitumour effect of the topoisomerase II poison to the central nervous system (CNS).

In another example, a topoisomerase II poison is used in the treatment of solid tumours. Again, the drug is administered systemically and penetrates the tumour, where the antiproliferative effect is desired. Again, the drug also circulates elsewhere in the body, with undesired deleterious effects. By also administering a topoisomerase II catalytic inhibitor which does not (or does not substantially) enter the acidic (low pH) microenvironment of solid tumours, those undesired deleterious effects can be reduced or eliminated, while not (or not substantially) affecting the desired antitumour effect in the solid tumour. In this way, the topoisomerase II catalytic inhibitor can be used as means of targeting the antitumour effect of the topoisomerase II poison to solid tumours (e.g., solid tumours characterised by an acid microenvironment).

Additionally, a topoisomerase II catalytic inhibitor can be used alone as a treatment of (e.g., accidental) extravasation of a topoisomerase II poison. For example, during administration, an injection of a topoisomerase II poison (e.g., as part of an anticancer therapy) may miss the vein so that the topoisomerase II poison “leaks” into the surrounding tissues, giving rise to accidental extravasation and associated tissue damage. In such cases, subsequent administration of a topoisomerase II catalytic inhibitor ameliorates the undesired effects (e.g., tissue damage) of the topoisomerase II poison associated with the accidental extravasation. The topoisomerase II catalytic inhibitor may be administered, for example, systemically (e.g., by injection into a vein) or locally (e.g., by injection into the tissue, e.g., the soft tissue, affected by the topoisomerase II poison extravasation, or by injection into the tissue, e.g., the soft tissue, at or near the location of topoisomerase II poison extravasation).

### Use in Methods of Inhibiting Topoisomerase II

One aspect of the present invention pertains to a method of inhibiting (e.g., catalytically inhibiting) topoisomerase II in a cell, in vitro or in vivo, comprising contacting the cell with an effective amount of a compound, as described herein.

In one embodiment, the method is performed in vitro.

In one embodiment, the method is performed in vivo.

In one embodiment, the compound is provided in the form of a pharmaceutically acceptable composition.

Suitable assays for determining topoisomerase II inhibition are described herein.

### Use in Methods of Therapy

Another aspect of the present invention pertains to a compound as described herein for use in a method of treatment of the human or animal body by therapy.

Another aspect of the present invention pertains to a compound as described herein for use in combination with a topoisomerase II poison, such as an anthracycline or an epipodophyllotoxin, in a method of treatment of the human or animal body by therapy.

Another aspect of the present invention pertains to a method of targeting the cytotoxicity of a topoisomerase II poison, comprising administering a compound as described herein, in combination with said topoisomerase II poison.

In one embodiment, the targeting is targeting to a solid tumour (e.g., the acid microenvironment of a solid tumour).

In one embodiment, the targeting is targeting to the central nervous systems (CNS) (e.g., the brain).

Another aspect of the present invention pertains to a method of permitting increased dosage of a topoisomerase II poison in therapy, comprising administering a compound as described herein, in combination with said topoisomerase II poison.

### Use in the Manufacture of Medicaments

Another aspect of the present invention pertains to use of a compound, as described herein, in the manufacture of a medicament for use in treatment.

Another aspect of the present invention pertains to use of a compound, as described herein, in the manufacture of a medicament for use in combination with a topoisomerase II poison, such as an anthracycline or an epipodophyllotoxin, in treatment.

### Methods of Treatment

Another aspect of the present invention pertains to a method of treatment comprising administering to a patient in need of treatment a therapeutically effective amount of a compound as described herein, preferably in the form of a pharmaceutical composition.

Another aspect of the present invention pertains to a method of treatment comprising administering to a patient in need of treatment a therapeutically effective amount of a compound as described herein, preferably in the form of a pharmaceutical composition, and a topoisomerase II poison, such as an anthracycline or an epipodophyllotoxin.

### Conditions Treated—Generally

In one embodiment (e.g., of use in methods of therapy, of use in the manufacture of medicaments, of methods of treatment), the treatment is treatment of a disease or condition that is ameliorated by the catalytic inhibition of topoisomerase II (e.g., a disease or condition that is known to be treated by topoisomerase II catalytic inhibitors).

### Conditions Treated—Proliferative Conditions and Cancer

In one embodiment (e.g., of use in methods of therapy, of use in the manufacture of medicaments, of methods of treatment), the treatment is treatment of a proliferative condition.

The terms “proliferative condition,” “proliferative disorder,” and “proliferative disease,” are used interchangeably herein and pertain to an unwanted or uncontrolled cellular proliferation of excessive or abnormal cells that is undesired, such as, neoplastic or hyperplastic growth.

In one embodiment, the treatment is treatment of a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, or lymphoma.

In one embodiment, the treatment is treatment of:

a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);

a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;

a tumour of mesenchymal origin, for example fibrosarcoma or habdomyosarcoma;

a tumour of the central or peripheral nervous system, for example astrocytoma, neuroblastoma, glioma or schwannoma;

melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

In one embodiment, the treatment is treatment of a proliferative condition of the central nervous system (CNS).

In one embodiment, the treatment is treatment of a tumour of the central nervous system (CNS).

In one embodiment, the treatment is treatment of brain cancer.

Conditions Treated—Damage associated with Extravasation

In one embodiment (e.g., of use in methods of therapy, of use in the manufacture of medicaments, of methods of treatment), the treatment is prevention or treatment of tissue damage (e.g., soft tissue damage) associated with extravasation of a topoisomerase II poison.

In one embodiment, the treatment is prevention or treatment of tissue damage associated with extravasation of a topoisomerase II poison in a patient receiving treatment with said topoisomerase II poison.

In one embodiment, the medicament is for systemic administration (i.e., is administered systemically) (e.g., by injection into a vein).

In one embodiment, the medicament is for local administration (i.e., is administered locally) (e.g., by injection into the tissue affected by the topoisomerase II poison extravasation, or by injection into the tissue at or near the location of topoisomerase II poison extravasation).

### Topoisomerase II Poisons

As discussed herein, the compounds described are useful in combination with topoisomerase II poisons. Many topoisomerase II poisons are known.

In one embodiment, the topoisomerase II poison is an anthracycline or an epipodophyllotoxin.

Examples of anthracyclines include doxorubicin, idarubicin, epirubicin, aclarubicin, mitoxantrone, dactinomycin, bleomycin, mitomycin, carubicin, pirarubicin, daunorubicin, daunomycin, 4-iodo-4-deoxy-doxorubicin, N,N-dibenzyl-daunomycin, morpholinodoxorubicin, aclacinomycin, duborimycin, menogaril, nogalamycin, zorubicin, marcellomycin, detorubicin, annamycin, 7-cyanoquinocarcinol, deoxydoxorubicin, valrubicin, GPX-100, MEN-10755, and KRN5500.

Examples of epipodophyllotoxins include etoposide, etoposide phosphate, teniposide, tafluposide, VP-16213, and NK-611.

In one embodiment, the topoisomerase II poison is etoposide (also known as Eposin, Etophos, Vepesid, VP-16).

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis, prevention) is also included. For example, use with patients who have not yet developed the condition, but who are at risk of developing the condition, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc., including a topoisomerase II poison, such as an anthracycline or an epipodophyllotoxin. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets. The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes.

The agents (i.e., the compound described herein, plus one or more other agents) may be formulated together in a single dosage form, or alternatively, the individual agents may be formulated separately and presented together in the form of a kit, optionally with instructions for their use, as described below.

### Routes of Administration

The active compound or pharmaceutical composition comprising the active compound may be administered to a subject by any convenient route of administration, whether systemically/peripherally or topically/locally (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the active compound to be administered alone, it is preferable to present it as a pharmaceutical formulation (e.g., composition, preparation, medicament) comprising at least one active compound, as defined above, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers. Formulations may also suitably be provided in the form of a depot or reservoir.

The active compound may be dissolved in, suspended in, or admixed with one or more other pharmaceutically acceptable ingredients. The active compound may be presented in a liposome or other microparticulate which is designed to target the active compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the active compound in a flavored basis, usually sucrose and acacia or tragacanth. Pastilles typically comprise the active compound in an inert matrix, such as gelatin and glycerin, or sucrose and acacia. Mouthwashes typically comprise the active compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base.

Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the active compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the active compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the active compound is dissolved or suspended in a suitable carrier, especially an aqueous solvent for the active compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the active compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 μg to about 250 mg (more typically about 100 μg to about 25 mg) per kilogram body weight of the subject per day. Where the active compound is a salt, an ester, an amide, a prodrug, or the like, the amount administered is calculated on the basis of the parent compound and so the actual weight to be used is increased proportionately.

### Kits

One aspect of the present invention pertains to a kit comprising (a) a compound, as described herein, preferably provided as a pharmaceutical composition and in a suitable container and/or with suitable packaging; and (b) instructions for use, for example, written instructions on how to administer the active compound.

In one embodiment, the kit further comprises a topoisomerase II poison, preferably provided as a pharmaceutical composition and in a suitable container and/or with suitable packaging.

The written instructions may also include a list of indications for which the active ingredient is a suitable treatment.

### Other Uses

The compounds described herein may also be used as cell culture additives to regulate cell proliferation, etc.

The compounds described herein may also be used as part of an in vitro assay, for example, in order to determine whether a candidate host is likely to benefit from treatment with the compound in question.

The compounds described herein may also be used as a standard, for example, in an assay, in order to identify other active compounds, other anti-proliferative agents, other anti-cancer agents, etc.

### EXAMPLES

The following are examples are provided solely to illustrate the present invention and are not intended to limit the scope of the invention, as described herein.

### Biological Methods

### Drugs and Reagents

ICRF-187 (Cardioxane, from Chiron Group) was dissolved in sterile water. Etoposide was purchased from Bristol-Myers Squibb and was diluted further in sterile water. m-AMSA (Amekrin, Pfizer) was diluted in DMSO. NSC 35866 was supplied from the Drug Synthesis Chemistry Branch, Development Therapeutics Program, Division of Cancer Treatment and Diagnosis, National Cancer Institute, Bethesda, Md., USA, and was dissolved in DMSO. ³H-dATP, ³H-thymidine and ¹⁴C-thymidine were all purchased from Amersham. Azathioprine, 6-thioguanine, 6-thiopurine, 2-thiopurine, 2,6-dithiopurine, 6-methylthioguanine, O⁶-benzylguanine, NU 2058, O⁶-methylguanine, 6-chloroguanine, acyclovir and 9-benzylguanine were all purchased from Sigma-Aldrich and dissolved in DMSO.

Purification of ³H-Labelled Crithidia fasciculata Kinetoplast DNA Network Decatenation Substrate

³H labelled kDNA network was isolated from Crithidia fasciculata grown in the presence of ³H-labelled thymidine as described in Shapiro et al., 1999. The specific activity of the DNA was typically 5000-10,000 cpm/μg DNA.

Purification of Human Topoisomerase II α from Over Expressing Yeast Cells

Wild-type and Y165S mutant human topoisomerase II α was purified from over-expressing yeast cells as described in Wassermann et al., 1993, with modifications described in Wessel et al., 1999, and was purified to greater than 95% purity as judged by SDS-PAGE and Coomassie blue staining.

### Inhibition of Topoisomerase II DNA Strand Passage Assay (Decatenation Assay)

Topoisomerase II catalytic activity (DNA strand passage activity) was measured by using a filter-based kDNA decatenation assay as described in Jensen et al., 2002. Briefly, 200 ng ³H labelled kDNA isolated from C. fasciculata was incubated with increasing concentrations of drug in 20 μL reaction buffer containing 10 mM TRIS-HCl pH 7.7, 50 mM NaCl, 50 mM KCl, 5 mM MgCl₂, 1 mM EDTA, 15 μg/mL BSA and 1 mM ATP using two units of purified wild-type or Y165S mutant topoisomerase II α for 20 minutes at 37° C. (where one unit of activity is defined as the amount of enzyme required for complete decatenation in the absence of drug). After addition of 5× stop buffer (5% Sarkosyl, 0.0025% bromophenol blue, and 50% glycerol), unprocessed kDNA network and decatenated DNA mini-circles were separated by filtering, and the amount of unprocessed kDNA in each reaction was determined by scintillation counting.

### Topoisomerase II ATPase Assay

ATP hydrolysis by human topoisomerase II α was linked to the oxidation of NADH as described in Lindsley, 2001 and references cited therein. The reaction was monitored spectrophotometrically at 340 nm using a Bio-Tek EL808 Ultra Micro plate Reader connected to a computer with KC4 Software installed (Bio-Tek Instruments, U.S.). The change in absorbance was related to ADP production using A₃₄₀^{1M}=6220 cm⁻¹. The reactions were performed in 96-well plates (Microtest 96-well Clear Plate, BD Falcon, BD Biosciences, NJ, USA) at 37° C. in a total volume of 400 μL buffer containing 50 mM HEPES pH 7.5, 8 mM Mg(OAc)₂, 150 mM KOAc, 2.1 mM phosphoenolpyruvate, 0.195 mM NADH, and 3.75 U of pyruvate kinase/9 U of lactate dehydrogenase. This coupled ATPase assay is fully functional under all reaction conditions employed; doubling any component of the ATP regeneration system had no measurable effect on the rates of ATP hydrolysis, whereas doubling the topoisomerase concentration doubled the measured rate of ATP hydrolysis. ATP and DNA were present at 1 mM and 2.82 nM (corresponding to a bp:enzyme-dimer ratio of 425) respectively. After an initial equilibration period, the reaction was initiated by the addition of 17.65 nM topoisomerase II α, and ATP hydrolysis was followed for 60 minutes. The rate of ATP hydrolysis, V, was determined from the linear part of the curve.

### Topoisomerase II DNA Cleavage Assay

In order to determine the ability of NSC 35866 to increase the level of topoisomerase II-DNA covalent complexes on DNA in vitro, a new and highly sensitive topoisomerase II DNA cleavage assay having a numeric readout was developed. This assay is based on the principle that DNA bound to protein (and hence human topoisomerase II α) is removed from the water phase after phenol chloroform extraction, while naked DNA remains in the water phase. The DNA substrate is a 950 bp linear ³H-labelled DNA synthesized by PCR in the presence of ³H-dATP. The DNA sequence is derived from a cDNA sequence of human topoisomerase I. The primers used in the PCR amplification were: forward GM ATA CGA GAC TGC TCG GC and reverse TTA AAA CTC ATA GTC TTC ATC AG. The DNA fragment was isolated from unincorporated dNTPs by ethanol precipitation at 0.3 M NaCl, followed by washing in 70% ethanol. The specific activity of the fragment was typically 10,000-20,000 cpm/μg. Before starting the assay, a drug dilution series comprising 10× the final drug concentration was made. Reaction mixtures containing 100 ng of the 950 bp linear ³H-labelled DNA, 300 ng human topoisomerase II q, topoisomerase II cleavage buffer (10 mM TRIS-HCL pH 7.9, 50 mM NaCl, 50 mM KCl, 5 mM MgCl₂, 1 mM EDTA, 15 μg/mL BSA and 1 mM Na₂ATP), and increasing concentrations of drug in 50 μL reaction volumes were then incubated 10 minutes at 37° C. A “no topoisomerase II” sample and a “no drug” sample were always included as controls. Next, the cleavable complex was trapped by adding 5 μL 10% SDS. After vigorous vortexing, 45 uL TE buffer, pH=8.0, was added to obtain 100 μL per sample. 100 μL phenol:chloroform:isoamyl alcohol (25:24:1) equilibrated with TE buffer, pH=8.0, was then added, and the samples were vortexed vigorously for 30 seconds. Finally, the samples were centrifuged at 20,000 g for 2 minutes and 90 μL of the upper water phase was used for scintillation counting using 15 mL of Ultima gold scintillation fluid (Packard).

### Topoisomerase II Retention on DNA/Streptavidin Beads

An assay capable of measuring non-covalent complexes of topoisomerase II on closed circular DNA was performed as described in Morris et al., 2000, with modifications. When performing six reactions, 60 μL M280 streptavidin coated bead (Dynal A/S, Oslo, Norway) slurry corresponding to 600 μg beads was transferred to a 1.5 mL tube that was then placed in a Dynal MPC-E (magnetic particle concentrator) rack (Dynal A/S, Oslo, Norway) for 1 to 2 minutes until the beads had settled on the tube wall. The beads were then washed twice in the DNA binding solution supplied with the kilobase binding kit (Dynal A/S, Oslo, Norway) by repeating this step. Finally, the beads were re-suspended in 250 μL DNA binding solution. A preparation of biotin labelled plasmid DNA containing a 5-kb super coiled circular DNA molecule carrying 8 successive PNA (Peptide Nucleic Acid) linked biotin labels at one known position (pGeneGrip biotin blank vector, Gene Therapy Systems Inc., San Diego, Calif., USA) was made by mixing 220 μL distilled water and 30 μL biotinylated DNA. After mixing the beads and the DNA preparation, the sample was left overnight at room temperature under gentle agitation to assure optimal formation of the DynaBeads DNA complex. Next, the complex was washed twice in 480 μL wash buffer (10 mM TRIS-HCL, pH 7.5, 2 M NaCl, 1 mM EDTA), once in distilled water, and once in topoisomerase reaction buffer (10 mM TRIS-HCl, pH 7.9, 50 mM NaCl, 50 mM KCl, 5 mM MgCl₂, 1 mM EDTA, 165 g/mL BSA). Then, the beads were re-suspended in 600 μL topoisomerase II buffer and divided into 6 tubes. 100 μL reactions containing plasmid DNA coated DynaBeads, topoisomerase II buffer, 2 μg purified human topoisomerase If a and drugs were incubated for 30 minutes at 37° C. When included, ATP was present at 1 mM. Next, each reaction mix was washed six times in 500 μL 2 M KCl containing the same drug concentration used during the previous incubation by applying the Dynal MPC as described above. After the last wash, the tubes were centrifuged at 20,000 g for 1 minute, and excess washing solution was removed. Next, 20 μL loading buffer (4% SDS, 20% glycerol, 10% β-mercaptaethanol, 5 mM EDTA) was added and the samples were boiled for 10 minutes and subjected to SDS-PAGE for one hour using a 7% tris acetate PAGE gel. As a positive control, 2 μg human topoisomerase II α was always included. As negative control a “no drug sample” was always included. After electrophoresis at 15 V/cm for 60 minutes, the gel was washed three times in 50 mL distilled water and stained using GelCode Blue Straining Reagent (Pierce, Rockford, Ill., USA) as described by the manufacturer, and the gel was photographed.

### Cell Lines

Human small cell lung cancer (SCLC) OC—NYH (de Leij et al., 1985) and NCI-H69 cells (Cuttitta et al., 1981) were grown in RPMI-1640 medium supplemented with 10% fetal calf serum, 100 U/mL penicillin-streptomycin at 37° C. in a humidified atmosphere containing 5% CO₂ in the dark.

### Clonogenic Assay

Clonogenic assay was performed essentially as described in Jensen et al., 1993. OC—NYH cells were exposed to increasing concentrations of NSC 35866 for 20 minutes, and were then co-exposed to 20 μM etoposide and the same concentrations of NSC 35866 for 60 minutes. Cells were then plated in 0.3% agar in 6 cm petri dishes with sheep red blood cells as feeder layer in triplicate, and were incubated under the same conditions as described above. Plates were counted after 3 weeks.

### Alkaline Elution Assay

Alkaline elution assay was performed as described in Kohn et al., 1976 with modifications as described in Sehested et al., 1998. Briefly, to assess the ability of NSC 35866 to protect against etoposide-induced DNA breaks, cells were incubated with increasing concentrations of NSC 35866 for 10 minutes, before 3 μM etoposide was added to the samples. The cells were then co-incubated with 3 μM etoposide along with the same concentrations of NSC 35866 for 60 minutes. Some samples contained no etoposide in order to assess whether NSC 35866 induced DNA breaks by itself. After incubation with drug, cells were lysed and the DNA fragments eluted. DNA in the experimental OC—NYH cells was metabolically labelled by ¹⁴C-thymidine incorporation while DNA in the internal control L1210 cells was metabolically labelled by ³H-thymidine incorporation.

### Band Depletion Assay

Band depletion assay was performed essentially as described in Sehested et al., 1998. The amount of extractable topoisomerase II α was detected by the ECL detection method (Amersham, Buckinghamshire, United Kingdom). OC—NYH cells were exposed to increasing concentrations of NSC 35866 for one hour and total proteins were extracted at 0.3 M NaCl. For detection of topoisomerase II α, a polyclonal primary antibody (Bio Trend, Cologne, Germany) was used. Horseradish peroxidase linked anti-rabbit antibody (Amersham, Buckinghamshire, United Kingdom) was used as secondary antibody.

### Abbreviations

Acyclovir, 9-[(2-hydroxyethoxy)methyl]guanine; AGT, O⁶-alkylguanine-DNA alkyltransferase; Azathioprine, 6-(1-methyl-4-nitroimidazol-5-yl)thiopurine; BSA, bovine serum albumin; CDK, cycline-dependent kinase; DMSO, dimethyl sulfoxide; DTT, dithiothreitol; ECL, enhanced chemo luminescence; EDTA, ethylenediaminetetraacetic acid; Etoposide, 4′-demethylepipodophyllotoxin 9-(4,6-O-ethylidene-b-D-glucopyranoside); IC50, inhibitory concentration resulting in 50% decreased activity; ICRF-187, (+)-1,2-bis(3,5-dioxopiperazinyl-1-yl)propane; kDNA, kinetoplast DNA; m-AMSA; methanesulfone-m-anisidine-4′-[(9-acridinyl)amino] hydrochloride; MTD, maximum tolerated dose; NADH, β-nicotinamide adenine dinucleotide reduced dipotassium salt; NSC 35866, 2-amino-6-(phenylethylthio)-purine; NU 2058, O⁶-cyclohexylmethylguanine; PAGE, polyacrylamide gel electroforesis; SCLC, small cell lung cancer; SDS, sodium dodecyl sulphate; TE, TRIS-EDTA; TRIS, tris(hydroxymethyl)aminomethane.

### Summary of Results

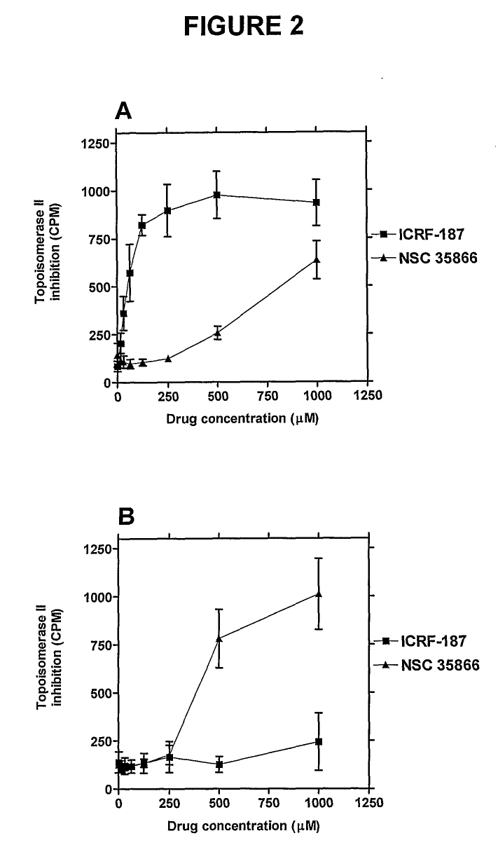

Initial screening results had shown that NSC 35866 inhibited the DNA strand passage activity of purified recombinant human topoisomerase II α. In order to establish a dose-response relationship for the inhibition of topoisomerase II DNA strand passage (catalytic) activity by NSC 35866, decatenation of Crithidia fasciculata kDNA network substrate was carried out as previously described (Jensen et al., 2002). FIG. 2 depicts the result of these experiments.

FIG. 2 describes the results of studies of the inhibition of topoisomerase II DNA strand passage activity by increasing concentrations of NSC 35866. Inhibition of human topoisomerase II α DNA strand passage activity was assessed by decatenation of tritium-labelled Crithidia fasiculata kDNA using a filter-based assay to separate unprocessed kDNA network from decatenated mini-circles. Panel A depicts the radioactivity and hence the amount of un-processed kDNA networks retained on the filter as a function of the concentration of ICRF-187 and NSC 35866 in the reactions as seen with wild-type human topoisomerase II α. Panel B depicts the inhibitory activity of these drugs as seen with bisdioxopiperazine resistant Y165S mutant human topoisomerase II α. Error bars represent SEM of three independent experiments in panel A and two independent experiments in panel B.

NSC35866 inhibited the DNA strand passage activity of wild-type human topoisomerase II α at concentrations above 250 μM, but was clearly less potent in comparison with the reference compound ICRF-187 (FIG. 2-A). The ability of NSC 35866 to inhibit the catalytic activity of Y165S mutant human topoisomerase II α was tested and showed no inhibition by bisdioxopiperazines including ICRF-187 (Wessel et al., 2002). While ICRF-187 was incapable of inhibiting the catalytic activity of the Y165S protein as expected, NSC 35866 was capable of doing so (FIG. 2-B). Interestingly, the Y165S protein appeared to be more sensitive towards inhibition by NSC 35866 than the wild-type protein (compare panels A and B in FIG. 2) suggesting that NSC 35866 may interact with topoisomerase II at the nucleotide-binding site.

The decatenation experiments described above (FIG. 2) indicate that NSC 35866 may interact with topoisomerase II at the nucleotide-binding site. If so, NSC 35866 would be expected to inhibit the ATPase reaction of topoisomerase II. To address this directly, the ability of NSC 35866 to inhibit the ATP hydrolysis reaction of purified recombinant human topoisomerase II α was assessed.

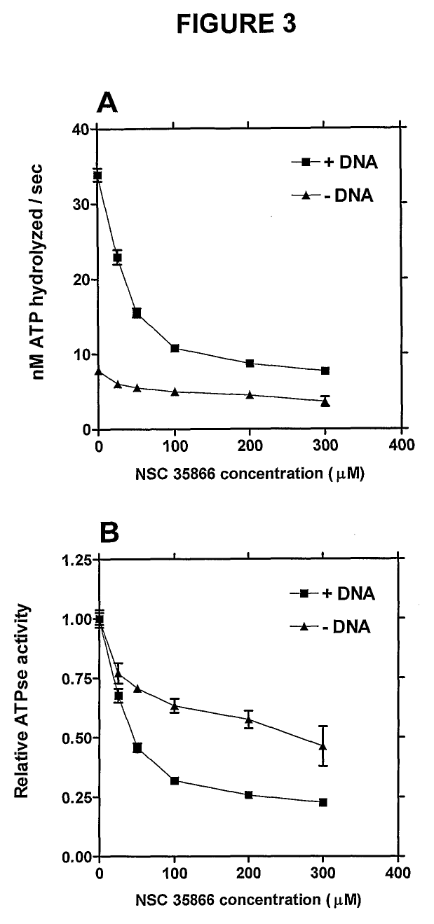

FIG. 3 describes the results of studies of the inhibition of human topoisomerase II α ATPase activity in the presence and absence of DNA by increasing concentrations of NSC 35866. The steady-state rate of ATP hydrolysis was determined using a coupled ATPase assay as described herein. Panel A depicts the absolute rates of ATP hydrolysis obtained in the absence of DNA and in the presence of plasmid DNA added at a base-pair to enzyme-dimer ratio of 425, plotted against increasing concentrations of NSC 35866. Panel B depicts the same data where the rate of ATP hydrolysis in the absence of NSC 35866 is normalized to one. This presentation allows for a direct comparison of the relative inhibition of ATPase activity by NSC 35866 in the absence and presence of DNA. Error bars represent SEM of two independent experiments each performed in duplicate.

Topoisomerase II is a DNA stimulated ATPase (Hammonds and Maxwell, 1997; Harkins and Linsley, 1998). In order to obtain a high signal in ATPase assay, the effect of NSC 35866 on ATPase activity in the presence of DNA was first investigated as described above. Under these conditions, the rate of ATP hydrolysis by human topoisomerase II α in the absence of drug was 35 nM ATP hydrolysed/sec (FIG. 3A). In the presence of DNA, NSC 35866 inhibited the rate of ATP hydrolysis with an IC₅₀ of 50 μM while 300 μM NSC 35866 inhibited 75% of the total ATPase activity (FIGS. 3A and B). Without DNA, the rate of ATP hydrolysis was 7.5 nM ATP hydrolysed/sec (FIG. 3A). NSC 35866 could also inhibit the DNA-independent ATPase activity, but without DNA the IC₅₀ value was increased to 300 μM (FIGS. 3A and B), suggesting that NSC 35866 targets mainly the DNA-bound conformation of topoisomerase II. Despite the fact that NSC 35866 seems to target mainly the DNA-bound configuration of topoisomerase II, its dependency on DNA for inhibition of topoisomerase II ATPase activity was much less pronounced than that seen for ICRF-187. In a similar ATPase assay the IC₅₀ value for ATPase inhibition by ICRF-187 was 1 μM in the presence of DNA while in the absence of DNA, 100 μM ICRF-187 was only capable of reducing the ATPase activity down to 75% of that seen in the absence of drug (data not shown). These results suggest that NSC 35866 and bisdioxopiperazines are likely to inhibit topoisomerase II by different mechanisms.

In order to understand in greater detail the mechanism of inhibition of NSC 35866 with human topoisomerase II α, structure-activity ATPase studies were performed. In these studies, the level of ATPase activity in the absence of drug was set to one. Two C9-substituted purine analogs, 9-benzylguanine and acyclovir (the latter being an inhibitor of viral DNA polymerase (Kleymann, 2003), had no inhibitory effect on the ATPase reaction of human topoisomerase II α at concentrations up to 300 μM (data not shown). 6-chloroguanine had also no inhibitory effect on the topoisomerase II ATPase reaction (data not shown).

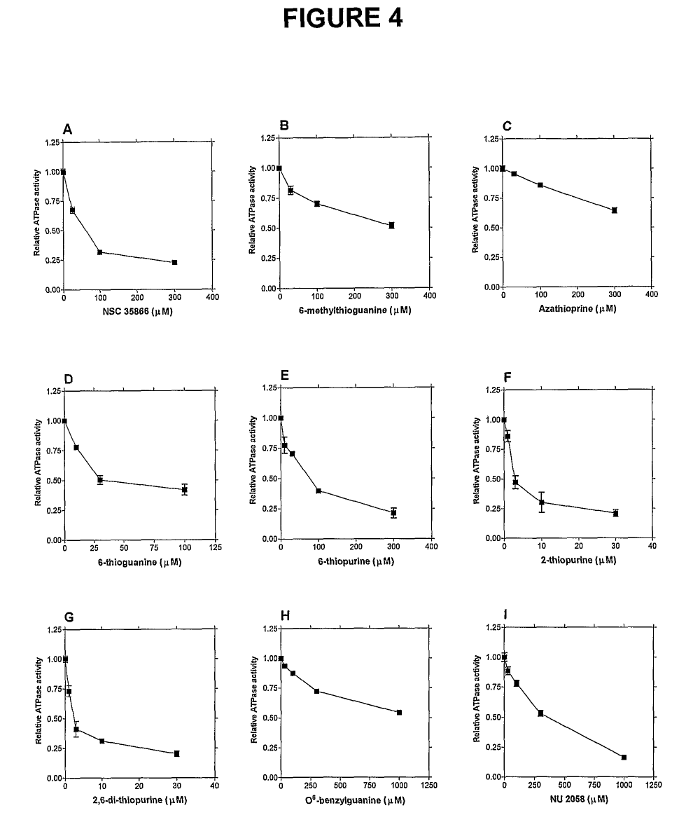

FIG. 4 describes the results of studies of the inhibition of human topoisomerase II α DNA-stimulated ATPase activity by various substituted purine analogs. The steady-state rate of ATP hydrolysis was determined as described in for FIG. 3 and as described herein. In this analysis, the rate of ATP hydrolysis in the absence of drug was set to one in all experiments. Error bars represent SEM of 2 or 3 independent experiments each preformed in duplicate.

Since NSC 35866 is a S⁶-substituted thio-ether of guanine, the ability of two other S⁶-substituted thio-ether purine analogs, 6-methylthioguanine and azathioprine (the latter being used as an anti-metabolite pro-drug in the clinic, see, e.g., Cara et al., 2004), to inhibit the topoisomerase II ATPase reaction was also assessed. Both compounds were capable of inhibiting topoisomerase II ATPase activity (FIG. 4B-C) but both were less potent than NSC 35866 (FIG. 3 and FIG. 4A).

To establish whether oxygen-based ether analogs may also work as topoisomerase II ATPase inhibitors, a series of O⁶-substituted guanine analogs were also tested for ability to inhibit topoisomerase II ATPase activity, namely O⁶-methylguanine, O⁶-benzylguanine (an inhibitor of the DNA repair protein AGT (Dolan and Pegg, 1997), and NU 2058 (an inhibitor of CDK1 and 2 (Hardcastle et al., 2004). NU 2058 can be regarded as an analog of O⁶-benzylguanine where the benzyl group has been substituted by the more flexible cyclohexane group. While O⁶-methylguanine had no detectable inhibitory effect on topoisomerase II ATPase activity at concentrations up to 300 μM (data not shown), O⁶-benzylguanine (FIG. 4H) and NU 2058 (FIG. 4I) were both active, having IC₅₀ values of 1000 and 300 μM respectively, thus being less active that NSC 35866 whose IC₅₀ is between 30 and 100 μM (FIG. 4A).

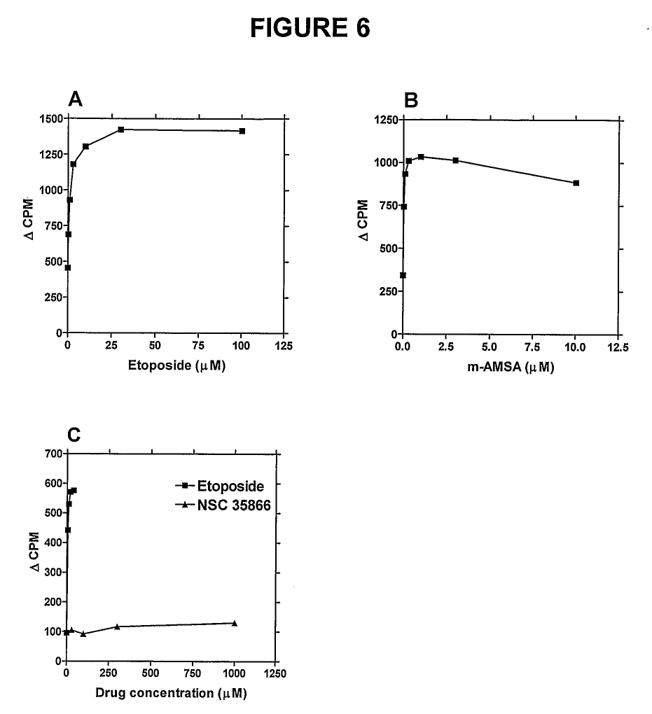

The effect of four different thiopurines with free SH groups, namely 6-thiogianine, 6-thiopurine, 2-thiopurine and 2,6-dithiopurine, were also tested as topoisomerase II ATPase inhibitors (6-thioguanine and 6-thiopurine are both used clinically as anti-metabolites, see, e.g., Cara et al., 2004). 6-thiopurine and 6-thioguanine both inhibited the ATPase activity of topoisomerase II, 6-thioguanine having an IC₅₀ around 30 μM (FIG. 4D) and 6-thiopurine having an IC₅₀ around 100 μM (FIG. 4E). 2-thiopurine and 2,6-dithiopurine inhibited topoisomerase II ATPase activity having IC₅₀ values around 3 μM (FIG. 4E-F).

A number of 6-thiopurine compounds were tested in topoisomerase II ATPase assay (measured in the Absence of DTT). The resulting IC₅₀ values are shown in the following table.

TABLE 1
IC50 Values for 6-Thiopurine Analogs in the Topoisomerase
II ATPase Assay, Measured in the Absence of DTT

| No. | NSC Drug | IC50 (μM) |
|---|---|---|
| 1 | NSC348401 | 0.372 |
| 2 | NSC348400 | 0.389 |
| 3 | NSC348402 | 0.777 |
| 4 | NSC244708 | 2.74 |
| 5 | NSC42375 | 7.87 |
| 6 | NSC52383 | 12.7 |
| 7 | NSC15747 | 13.4 |
| 8 | NSC46384 | 14.1 |
| 9 | NSC39331 | 19 |
| 10 | NSC38732 | 34.06 |
| 11 | NSC52388 | 36.4 |
| 12 | NSC35865 | 53.56 |
| 13 | NSC36824 | 68.9 |
| 14 | NSC35862 | 85.4 |
| 15 | NSC647471 | 118.6 |
| 16 | NSC172614 | 120.7 |
| 17 | NSC39328 | 151.2 |

Recombinantly expressed human topoisomerase II α purified by a protocol similar to the one used here has been shown to contain free cysteine residues (Hasinoff et al., 2004). Furthermore, thiopurines having free SH functionalities have been shown to covalently modify proteins at free cysteine residues (Mojena et al., 1992). The ability of all active compounds to inhibit topoisomerase II ATPase activity was tested in the presence of 10 mM DTT, because DTT is expected to inhibit the formation of thiopurine-topoisomerase II covalent interactions. While NSC 35866, O⁶-benzylguanine and NU 2058 could inhibit ATPase activity when DTT was present in the reaction buffer, this was not the case with the four thiopurines having free SH functionalities (data not shown). This result suggests that thiopurines with free SH groups inhibit topoisomerase II ATPase activity by covalently modifying free cysteine residues, while NSC 35866, O⁶-benzylguanine and NU 2058 work by non-covalent interactions in accordance with their expected reactivity.

In order to ensure that the experimental compounds inhibited ATP hydrolysis by interacting with human topoisomerase II α, and not by interfering with the lactate dehydrogenase and pyruvate kinase coupling enzymes also present in the ATPase reaction, the following control experiments were performed. In ATPase reactions containing fixed concentrations of inhibitory purines resulting in 50-80% inhibition of ATP hydrolysis under standard conditions (depending on the potency of the compound), the amount of topoisomerase II was increased 3- and 6-fold. If the experimental compounds work by inhibiting topoisomerase II α and not by inhibiting the coupling enzymes, increasing the amount of topoisomerase II should increase the rate of ATP hydrolysis by a similar factor, which was indeed the case (data not shown). Furthermore, if the experimental compounds decrease ATP hydrolysis by inhibiting topoisomerase II and not by inhibiting the coupling enzymes, increasing the level of the coupling enzymes in the presence of fixed concentrations of drug should have little or no effect on the rate of ATP hydrolysis, which was also the case (data not shown). Together, these control experiments demonstrate that these purine analogs do in fact work as inhibitors of the ATPase reaction of human topoisomerase II α.

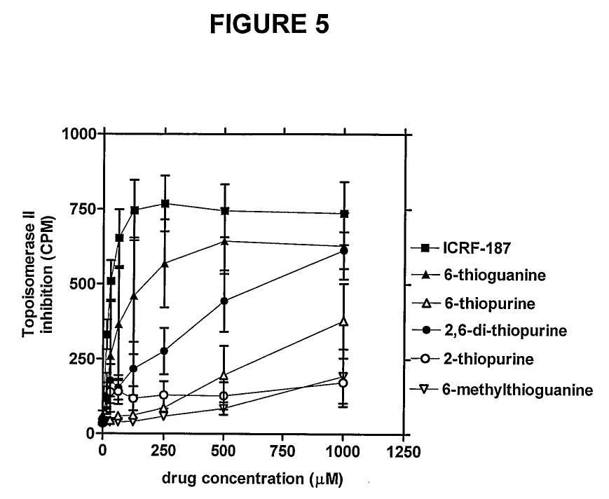

Since some of the thiopurines used in the ATPase structure-activity studies above are used as anti-metabolites in the clinic (6-thioguanine, 6-thiopurine, and azathioprine, which is a pro-drug of the latter, see, e.g., Cara et al., 2004), it would be interesting to determine their inhibitory action on the DNA strand passage reaction of human topoisomerase II α. The results of these experiments are shown in FIG. 5.

FIG. 5 shows the results of studies of the inhibition of human topoisomerase II α DNA strand passage activity by selected thiopurines. Inhibition of human topoisomerase II α DNA strand passage activity was determined by decatenation of tritium labelled Crithidia fasiculata kDNA as described for FIG. 2. Error bars represent SEM of 3 or 4 independent experiments.

In this analysis, 6-thioguanine inhibited the catalytic activity of topoisomerase II. Although this compound did not reach a maximal level of inhibition similar to that of the reference compound ICRF-187, it displayed a rapid onset and half-maximal inhibition was achieved around 50 μM. 6-thiopurine was much less potent, and maximal inhibition was apparently not reached at 1000 μM (FIG. 5), suggesting that the NH₂ group present only in 6-thioguanine plays a role for topoisomerase II inhibition. 2-thiopurine and 2,6-dithiopurins were both less potent in inhibiting topoisomerase II DNA strand passage activity than 6-thioguanine (FIG. 5) despite the fact that these compounds were more potent than 6-thioguanine in their inhibition of topoisomerase II ATPase activity (compare FIG. 4D to FIG. 4F-G). 2-thiopurine had virtually no effect while 2,4-dithiopurine had an effect between that of the two 6-substituted thiopurines (FIG. 5). Together the results presented in FIG. 4 and FIG. 5 indicate that specific types of cystein modifications may have differential effects on the ATPase- and DNA strand passage reactions of human topoisomerase II α. In accordance with its weak effect in the ATPase assay, 6-methylthioguanine showed almost no inhibition of decatenation activity.

The results presented herein show that NSC35866 targets topoisomerase II in vitro with a mode of interaction different of that of the bisdioxopiperazines.

In order to establish whether NSC 35866 inhibits the DNA strand passage reaction of topoisomerase II by stabilising a covalent reaction intermediate, a new and highly sensitive topoisomerase II DNA cleavage assay having a numeric read-out was developed. This assay is based on the fact that after extraction with phenol-chloroform, protein-bound DNA is removed from the water phase, while naked DNA remains in the water phase. The covalent topoisomerase II-DNA complex is a DNA-protein complex. Consequently, in reactions containing topoisomerase II and linear DNA, the ability of compounds to remove DNA from the water phase after phenol-chloroform extraction should reflect their potency as topoisomerase II poisons. This assay was first validated by incubating 100 ng of a linear 950 bp PCR DNA fragment with 300 ng of purified human topoisomerase II α in the presence of increasing concentrations of the etoposide and m-AMSA. The DNA fragment was ³H labelled by performing PCR in the presence of ³H-dATP. In these experiments, a “no topoisomerase II” sample was always included to determine the level of radioactivity (DNA) retained in the water-phase when no enzyme is present. Within each experiment, the CPM values retained in the water phase in the topoisomerase II reactions were then subtracted from this background CPM value to give Δcpm. Consequently, the Δcpm values of samples with no drug added represent the background level of topoisomerase II-DNA covalent complexes present in the reaction mixture under the assay conditions, while the Δcpm levels in the presence of drugs represent the levels of poison-induced topoisomerase II-DNA covalent complexes.

FIG. 6 describes the results of studies of the lack of stimulation of the level of human topoisomerase II α-DNA covalent complexes by NSC 35866. A novel and highly sensitive method of determining the level of topoisomerase II-DNA covalent complexes based on phenol-chloroform extraction as described herein was employed. Panel A depicts increased levels of human topoisomerase II α covalent complexes with DNA as function of increasing concentrations of etoposide, while Panel B depicts covalent complex formation as function of increasing concentrations of m-AMSA. Panel C depicts the effect of increasing concentrations of NSC 35866 at concentrations up to 1000 μM, with etoposide (up to 40 μM) included as positive control. While etoposide increased the level of covalent complex formation by a factor of 6, there was no measurable effect of 1000 μM NSC 35866, showing that NSC 35866 is not a topoisomerase II poison.

FIG. 6A depicts Δcpm as the function of increasing concentrations of etoposide while FIG. 6B depicts Δcpm as the function of increasing levels of m-AMSA. Both drugs increase Δcpm in a dose-dependent manner as expected. The assay was also carried out in the presence of increasing concentrations of etoposide while omitting ATP from the reaction. Under these conditions, no detectable increase in Δcpm was observed (data not shown), in accordance with published data that ATP is required for etoposide to efficiently induce DNA cleavage (Wang et al., 2001). Together, these data demonstrate that this assay is actually measuring the level of topoisomerase II covalent cleavage complexes on DNA.

The ability of NSC 35866 to increase the level of topoisomerase II-DNA covalent complexes was next tested using etoposide as a positive control (FIG. 6C). While etoposide was found to increase Δcpm efficiently, NSC 35866 had no effect on the level of covalent cleavage complex formation at concentrations up to 1000 μM, showing that NSC 35866 is not a topoisomerase II poison. The ability of NSC 35866 to inhibit the DNA strand passage reaction of topoisomerase II without increasing the level of the cleavage complex establishes that this compound is a catalytic topoisomerase II inhibitor.

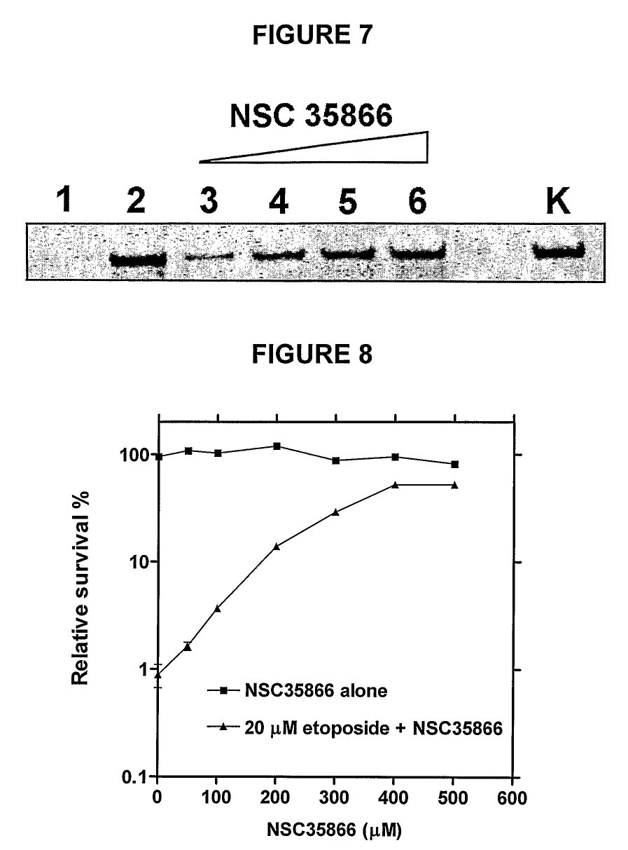

Bisdioxopiperazines are known to stabilise a salt-stable protein clamp of topoisomerase II on circular closed DNA whose formation depends on ATP (see, e.g., Morris et al., 2000; Renodon-Corniere et al., 2002; Roca et al., 1994). The ability of NSC 35866 to induce a salt-stable complex of human topoisomerase II α around circular DNA was next assessed. In order to do so, an assay measuring the retention of topoisomerase II on circular plasmid DNA attached to magnetic beads via biotin-streptavidin linkage was used, as described in Morris et al., 2000 and as described above. FIG. 7 depicts the result of a typical experiment.

FIG. 7 describes the results of studies of the ability of NSC 35866 to stabilise a salt-stable complex of human topoisomerase II α on covalently closed circular DNA. Retention of salt-stable (to 2 M KCl) complexes of human topoisomerase II α on circular DNA attached to magnetic beads via biotin-streptavidin linkage was determined by eluting retained protein by adding running buffer containing 4% SDS followed by heating to 100° C. for 10 minutes. The amount of human topoisomerase II α protein retained was then determined by running the samples on 7% SDS-PAGE gels followed by straining with GelCode Blue Strain Reagent (Pierce, Rockford, Ill., USA): Lane 1, no drug; Lane 2, 200 μM ICRF-187; Lane 3, 30 μM NSC 35866; Lane 4, 100 μM NSC 35866; Lane 5, 300 μM NSC 35866; Lane 6, 1000 μM NSC 35866; Lane K, 2 μg human topoisomerase II α. FIG. 7 depicts representative data of four independent experiments.

In the absence of any drug, very little protein was retained on the beads after washing at 2 M KCl (FIG. 7, Lane 1). Addition of 200 μM ICRF-187 to the reaction mixture strongly induced the retention of topoisomerase II to the beads (FIG. 7, Lane 2). FIG. 7, Lanes 3-6 depict protein retention in the presence of increasing concentrations of NSC 35866 (30, 100, 300 and 1000 μM). It is evident that NSC 35866 traps human topoisomerase II α as a salt-stable complex on circular closed DNA in a dose-dependent manner. NSC 35866 was also capable of trapping the protein as a salt-stable closed clamp on DNA in the absence of ATP, in three repeated experiments but only at 300 and 1000 μM, indicating that trapping is less efficient in the absence of the ATP cofactor (data not shown). In contrast, protein retention induced by ICRF-187 strongly depended on ATP (data not shown).

Several structurally unrelated topoisomerase II catalytic inhibitors including the bisdioxopiperazines have the capacity of protecting cells from cytotoxicity induced by exposure to topoisomerase II poisons (see, e.g., Jensen et al., 1997; Jensen et al., 1990; Hasinoff et al., 1996; Ishida et al., 1996; Sehested et al., 1993, Jensen et al., 1994). The ability of NSC 35866 to rescue human cancer cells from etoposide-induced cytotoxicity was tested. Pre-exposure of human SCLC OC—NYH cells to increasing concentrations of NSC 35866 for 20 minutes followed by co-exposure for 60 minutes could antagonise etoposide-induced cytotoxicity in a dose-dependent manner. A typical experiment of three is depicted in FIG. 8.

FIG. 8 describes the results of studies of the ability of NSC 35866 to efficiently antagonise cytotoxicity induced by a one-hour exposure of human SCLC cells to 20 μM etoposide in a dose-dependent manner. OC—NYH cells were first pre-incubated for 20 minutes with increasing concentrations of NSC 35866. 20 μM etoposide was then added, and the cells were incubated for one hour. Next, the drugs were washed out and the cells were plated and counted after three weeks as described herein. The relative survival of cells receiving the various treatments as compared to cells receiving no treatment was finally plotted against NSC 35866 concentration. FIG. 8 depicts representative data of three experiments.

It is evident that NSC 35866 is capable of reducing cytotoxicity induced by a one-hour treatment with 20 μM etoposide in a dose-dependant manner. NSC 35866 was capable of reducing etoposide-induced cytotoxicity up to 50 fold. NSC 35866 was likewise capable of protecting human SCLC NCI-H69 cells from etoposide-induced cytotoxicity (data not shown). These data demonstrate that NSC 35866 functions as a catalytic inhibitor of topoisomerase II in human cells. The ability of other purine analogs to inhibit etoposide-induced cytotoxicity with human SCLC OC—NYH cells was also tested. The effect of 6-thiopurine and 6-thioguanine at concentrations up to 300 μM, the effect of azathioprine and 6-methylthioguanine at concentrations up to 500 μM, and the effect of 2-thiopurine and 2,6-dithiopurine at concentrations up to 30 μM, was also tested, and no detectable effect on the level of etoposide-induced cytotoxicity was observed (data not shown). The finding that 6-thioguanine has no effect on etoposide-induced cytotoxicity at 300 μM—a concentration at which NSC 35866 is highly protective—while 6-thioguanine is more potent in inhibiting the DNA strand passage reaction of topoisomerase II in vitro than NSC 35866, confirms the notion that thiopurines having free SH functionalities inhibit topoisomerase II with a mechanism of action different from that of NSC 35866.

The alkaline elution assay represents a direct and highly sensitive way of measuring DNA breaks in cells (see, e.g., Kohn et al., 1976). Because the assay is performed at alkaline pH, the sum of DNA single strand breaks and DNA double strand breaks is detected. The alkaline elution assay was used to study the mechanism of NSC 35866-induced antagonism etoposide.

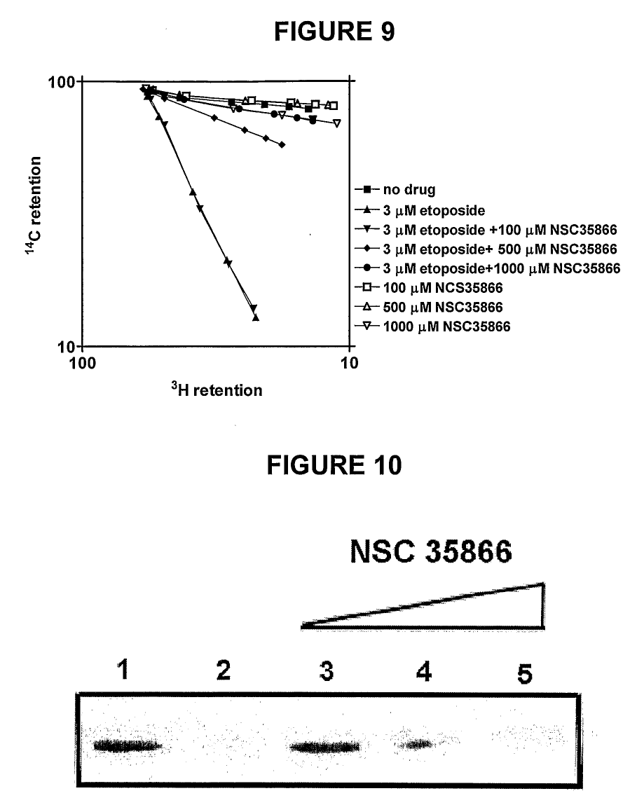

FIG. 9 describes the results of studies of the ability of NSC 35866 to antagonise DNA breaks induced by etoposide in human SCLC OC—NYH cells in a dose dependent manner. Alkaline DNA elution was used to detect DNA fragmentation induced by 3 μM etoposide in the presence of increasing concentrations of NSC 35866 as described herein. H₂0₂ treated mouse leukemic L1210 cells were used as internal control for DNA fragmentation. The DNA of the experimental OC—NYH cells was ¹⁴C-labelled while the DNA of the L1210 cells was 3H-labelled. While NSC 35866 does not result in increased DNA fragmentation when applied alone, this compound is clearly capable of antagonising the effect of etoposide in a dose-dependent manner.

FIG. 9 depicts the result of an alkaline elution assay. It is evident that 3 μM etoposide results in extensive fragmentation of DNA. Although 100 μM NSC 35866 had no detectable effect on the level of etoposide-induced DNA breaks, 500 μM NSC 35866 partly antagonised the effect of etoposide, while 1000 μM NSC 35866 completely antagonised etoposide-induced DNA breaks. From FIG. 9 it is also evident that NSC 35866 does not induce detectable levels of DNA breaks by itself at concentrations up to 1000 μM in accordance with the DNA cleavage results (FIG. 6C). Due to the lack of effect of 100 μM NSC 35866 on etoposide-induced DNA breaks, the alkaline elution assay was repeated using 30, 100 and 300 μM NSC 35866. While 30 and 100 μM NSC 35866 had no detectable effect on the levels of DNA breaks induced by 3 μM etoposide, 300 μM NSC 35866 partly antagonised the effect of etoposide (data not shown).

The band depletion assay can be used to assess the binding of proteins to DNA in cells under various conditions (see, e.g., Kaufmann and Svingen, 1999). If a given compound increases the stability of a proteins' interaction with DNA, that protein becomes less extractable at 0.3 M NaCl. The finding that NSC 35866 is capable of inducing a salt-stable complex of human topoisomerase II α on DNA in vitro (FIG. 7) prompted the assessment of whether NSC 35866 treatment decreases the amount of human topoisomerase II α extractable from human SCLC OC—NYH cells.

FIG. 10 describes the results of studies of the ability of NSC 35866 to trap human topoisomerase II α as a non-extractable complex on DNA in a dose dependent manner. The ability of NSC 35866 to stabilise topoisomerase II α as a non-extractable complex on DNA in human SCLC OC—NYH cells was assessed using the band depletion assay as described herein. The amounts of topoisomerase II α was visualised by western blotting using a topoisomerase II α specific primary antibody: Lane 1, no drug; Lane 2, 200 μM ICRF-187; Lane 3, 200 μM NSC 35866; Lane 4, 500 μM NSC 35866; Lane 5, 1000 μM NSC 35866. Band depletion of the topoisomerase II α isoform caused by NSC 35866 was detected in two independent experiments.

FIG. 10 depicts the result of a band depletion assay measuring the extractable amount of human topoisomerase II α protein as determined by western blot. 200 μM ICRF-187 (FIG. 10, Lane 2) clearly reduced the amount of extractable topoisomerase II α compared to the “no drug” sample (FIG. 10, Lane 1) as expected. NSC 35866 also decreased the extractable amount of topoisomerase II α. While 200 μM NSC 35866 had no effect (FIG. 10, Lane 3), exposure of the cells to 500 (FIG. 10, Lane 4) and 1000 μM NSC 35866 (FIG. 10, Lane 5) reduced the amount of extractable topoisomerase II. Decreased amounts of extractable topoisomerase II α protein were detected in two independent experiments. These results suggest that NSC 35866 traps topoisomerase II α as a protein clamp around DNA in cells at concentrations where the drug inhibits etoposide-induced cytotoxicity and DNA breaks in human SCLC OC—NYH cells (compare FIG. 8, FIG. 9, and FIG. 10).

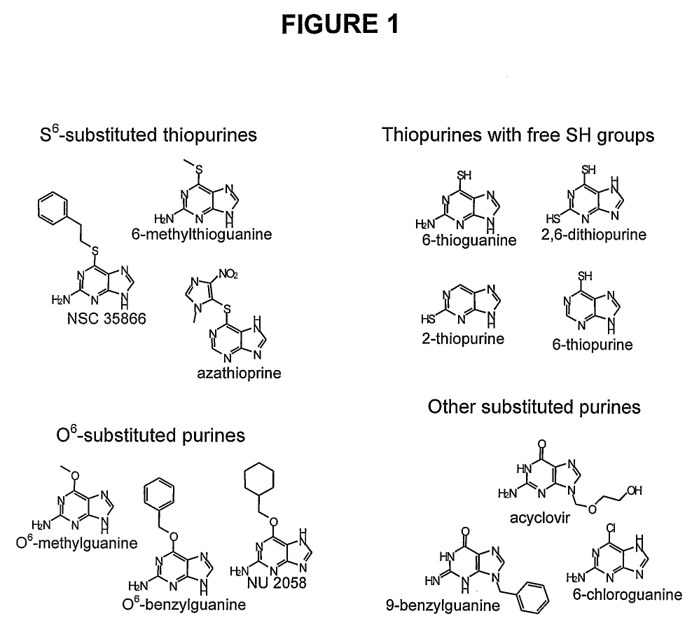

It is established herein that NSC 35866 functions as a catalytic inhibitor of topoisomerase II in vitro and in human cancer in cells. This compound inhibits topoisomerase II ATPase activity (FIG. 3) and DNA strand passage activity (FIG. 2) in vitro, without increasing the level of topoisomerase II-DNA covalent complex (FIG. 6). This compound also antagonizes etoposide-induced cytotoxicity (FIG. 8) and DNA breaks (FIG. 9) in human cancer cells. Furthermore, the data suggests that NSC 35866 inhibits topoisomerase II by a mechanism involving the stabilization of a closed clamp complex of topoisomerase II around DNA (FIG. 7 and FIG. 10). Structure activity studies establish that NSC 35866 belongs to a novel structural class of purine-based topoisomerase II catalytic inhibitors (FIG. 4). Although this mechanism of action is reminiscent of that of the bisdioxopiperazines (see, e.g., Morris et al., 2000; Renodon-Corniere et al., 2002; Roca et al., 1994), NSC 35866 is much less potent than these compounds in inhibiting human topoisomerase II α (FIG. 2). In addition, mutant topoisomerase II incapable of being inhibited by bisdioxopiperazines responds at least as well to inhibition by NSC 35866 as the wild-type protein (FIG. 2). This result indicates that NSC 35866 and the bisdioxopiperazines inhibit topoisomerase II by different mechanisms although similarities exist. This is also supported by the notion that NSC 35866 shows much less dependence on DNA for its inhibition of topoisomerase II ATPase activity (FIG. 3 and data not shown), and by the finding that NSC 35866 can stabilize a closed clamp complex on DNA even in the absence of ATP. The existence of these differences is possibly not surprising, given the lack of structural similarity between bisdioxopiperazines and NSC 35866 (FIG. 1). The bisdioxopiperazine-binding pocket (ICRF-187) on yeast topoisomerase II has recently been resolved by x-ray crystallography (see, e.g., Classen et al., 2003), and the drug binding site described in that work does not suggest that NSC 35866 interacts at this interaction site in agreement with the biochemical data described herein.

In order to obtain some insight into the mechanism of topoisomerase II ATPase inhibition by NSC 35866, a structure-activity study was performed including 12 other substituted purine analogs (FIG. 4). In this analysis NSC 35866 was capable of inhibiting topoisomerase II ATPase activity in the presence of DTT as opposed to thiopurines with free SH groups that were only active in the absence of DTT. This indicates that the latter inhibits topoisomerase II ATPase activity through covalent modification of free cysteine residues, a mechanism of protein interaction previously suggested for thiopurines having free SH functionalities (see, e.g., Mojena et al., 1992). NSC 35866 was highly efficient in protecting human cancer cells from etoposide-induced cytotoxicity (FIG. 8), while this was not the case for various thiopurines having free SH functionalities (data not shown). At least two explanations for this observation are contemplated: (i) covalent topoisomerase II cysteine modifications caused by thiopurines having free SH groups may not render topoisomerase II resistant towards the action of etoposide inside cells; and (ii) free SH groups in other cellular proteins may compete with those in topoisomerase II for covalent modification by thiopurines with free SH groups hereby abolishing their effect on topoisomerase II in cells. In any case, this result underscores the notion that NSC 35866 and thiopurines having free SH functionalities work by different mechanisms in cells.

Although NSC 35866 is clearly established as a catalytic inhibitor of topoisomerase II in vitro and in human cells, a number of drawbacks may preclude the use of this compound as pharmacological modulator of topoisomerase II poisons in its present form. First, the potency of NSC 35866 towards topoisomerase II in vitro and in cells is rather low, and high μM concentrations are required to obtain a response in all assays expect in the ATPase assay. Second, due to its purine structure, NSC 35866, or its possible in vivo hydrolysis product 6-thioguanine, is likely to be incorporated into DNA. If so, this would implicate NSC 35866 being both an anti-metabolite and a topoisomerase II catalytic inhibitor. Incorporation of 6-thioguanine into DNA has been shown to increase DNA cleavage by topoisomerase II (see, e.g., Krynetskaia et al., 2000), suggesting that in the case NSC 35866 is actually hydrolysed to 6-thioguanine in vivo followed by incorporation into DNA, a topoisomerase II poison-like mode of action could be the result.

ATPase structure-activity studies described herein establish that O⁶-substituted guanine analogs also have the capacity of inhibiting topoisomerase II. Here, results obtained with a series of O⁶-substituted analogs of guanine, namely O⁶-methylguanine, O⁶-benzylguanine, and NU 2058 (data not shown and FIG. 4 H-I), suggest that it may be possible to increase further the potency of O⁶-substituted purine analogs as topoisomerase II inhibitors. NU 2058 targets cell cycle progression (see, e.g., Hardcastle et al., 2004) while at the same time displaying activity against human topoisomerase II ATPase activity (FIG. 4I). Purine-based compounds that target topoisomerase II and cell cycle progression in concert would be very useful as anti-cancer agents.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

The present invention is not limited to those embodiments which are encompassed by the appended claims, which claims pertain to only some of many preferred embodiments.

### REFERENCES

A number of patents and publications are cited herein in order to more fully describe and disclose the invention and the state of the art to which the invention pertains. Full citations for these references are provided herein. Each of these references is incorporated herein by reference in its entirety into the present disclosure.
Andoh T and Ishida R, Catalytic inhibitors of DNA topoisomerase II. Biochim. Biophys. Acta 1400: 155-171, 1998.
Boritzki T J, Wolfard T S, Besserer J A, Jackson R C, and Fry D W, Inhibition of type II topoisomerase by fostriecin. Biochem. Pharmacol. 37: 4063-4068, 1988.
Cara C J, Pena A S, Sans M, Rodrigo L, Guerrero-Esteo M, Hinojosa J, Garcia-Paredes J, and Guijarro L G, Reviewing the mechanism of action of thiopurine drugs: towards a new paradigm in clinical practice. Med. Sci. Monit. 10: RA247-RA254, 2004.
Classen S, Olland S, and Berger J M, Structure of the topoisomerase II ATPase region and its mechanism of inhibition by the chemotherapeutic agent ICRF-187. Proc. Natl. Acad. Sci. U.S.A 100: 10629-10634, 2003.
Cuttitta F, Rosen S, Gazdar A F, and Minna J D, Monoclonal antibodies that demonstrate specificity for several types of human lung cancer. Proc. Natl. Acad. Sci. U.S.A 78: 4591-4595, 1981.
de Leij L, Postmus P E, Buys C H, Elema J D, Ramaekers F, Poppema S, Brouwer M, van der Veen A Y, Mesander G, and The T H, Characterization of three new variant type cell lines derived from small cell carcinoma of the lung. Cancer Res 45: 6024-6033, 1985.
Dolan M E and Pegg A E, 06-benzylguanine and its role in chemotherapy. Clin. Cancer Res. 3: 837-847, 1997.
Drake F H, Hofmann G A, Mong S M, Bartus J O, Hertzberg R P, Johnson R K, Mattern M R, and Mirabelli C K, In vitro and intracellular inhibition of topoisomerase II by the antitumor agent merbarone. Cancer Res. 49: 2578-2583, 1989.
Goto T and Wang J C, Yeast DNA topoisomerase II. An ATP-dependent type II topoisomerase that catalyzes the catenation, decatenation, unknotting, and relaxation of double-stranded DNA rings. J. Biol. Chem. 257: 5866-5872, 1982.
Hammonds T R and Maxwell A, The DNA dependence of the ATPase activity of human DNA topoisomerase II alpha. J. Biol. Chem. 272: 32696-32703, 1997.
Hardcastle I R, Arris C E, Bentley J, Boyle F T, Chen Y, Curtin N J, Endicott J A, Gibson A E, Golding B T, Griffin R J, Jewsbury P, Menyerol J, Mesguiche V, Newell D R, Noble M E, Pratt D J, Wang L Z, and Whitfield H J, N2-substituted O6-cyclohexylmethylguanine derivatives: potent inhibitors of cyclin-dependent kinases 1 and 2. J. Med. Chem. 47: 3710-3722, 2004.
Harkins T T and Lindsley J E, Pre-steady-state analysis of ATP hydrolysis by Saccharomyces cerevisiae DNA topoisomerase II. 1. A DNA-dependent burst in ATP hydrolysis. Biochemistry 37: 7292-7298, 1998.
Hasinoff B B, Wu X, Krokhin O V, Ens W, Standing K G, Nitiss J L, Sivaram T, Giorgianni A, Yang S, Jiang Y, and Yalowich J C, Biochemical and Proteomics Approaches to Characterize Topoisomerase II{alpha} Cysteines and DNA as Targets Responsible for Cisplatin-induced Inhibition of Topoisomerase II{alpha}. Mol. Pharmacol.—ahead of print, 2004.
Hasinoff B B, Yalowich J C, Ling Y, and Buss J L, The effect of dexrazoxane (ICRF-187) on doxorubicin- and daunorubicin-mediated growth inhibition of Chinese hamster ovary cells. Anticancer Drugs 7: 558-567, 1996.
Holm B, Jensen P B, and Sehested M, ICRF-187 rescue in etoposide treatment in vivo. A model targeting high-dose topoisomerase II poisons to CNS tumors. Cancer Chemother. Pharmacol 38: 203-209, 1996.
Holm B, Jensen P B, Sehested M, and Hansen H H, In vivo inhibition of etoposide-mediated apoptosis, toxicity, and antitumor effect by the topoisomerase II-uncoupling anthracycline aclarubicin. Cancer Chemother. Pharmacol. 34: 503-508, 1994.
Holm B, Sehested M, and Jensen P B, Improved targeting of brain tumors using dexrazoxane rescue of topoisomerase II combined with supralethal doses of etoposide and teniposide. Clin. Cancer Res 4: 1367-1373, 1998.
Ishida R, Iwai M, Hara A, and Andoh T, The combination of different types of antitumor topoisomerase II inhibitors, ICRF-193 and VP-16, has synergistic and antagonistic effects on cell survival, depending on treatment schedule. Anticancer Res 16: 2735-2740, 1996.
Ishida R, Miki T, Narita T, Yui R, Sato M, Utsumi K R, Tanabe K, and Andoh T, Inhibition of intracellular topoisomerase II by antitumor bis(2,6-dioxopiperazine) derivatives: mode of cell growth inhibition distinct from that of cleavable complex-forming type inhibitors. Cancer Res 51: 4909-4916, 1991.
Jensen L H, Renodon-Corniere A, Wessel I, Langer S W, Sokilde B, Carstensen E V, Sehested M, and Jensen P B, Maleimide is a potent inhibitor of topoisomerase II in vitro and in vivo: a new mode of catalytic inhibition. Mol Pharmacol 61: 1235-1243, 2002.
Jensen P B and Sehested M, DNA topoisomerase II rescue by catalytic inhibitors: a new strategy to improve the antitumor selectivity of etoposide. Biochem Pharmacol 54: 755-759, 1997.
Jensen P B, Sorensen B S, Demant E J, Sehested M, Jensen P S, Vindelov L, and Hansen H H, Antagonistic effect of aclarubicin on the cytotoxicity of etoposide and 4′-(9-acridinylamino)methanesulfon-m-anisidide in human small cell lung cancer cell lines and on topoisomerase II-mediated DNA cleavage. Cancer Res. 50: 3311-3316, 1990.
Jensen P B, Sorensen B S, Sehested M, Demant E J, Kjeldsen E, Friche E, and Hansen H H, Different modes of anthracycline interaction with topoisomerase II. Separate structures critical for DNA-cleavage, and for overcoming topoisomerase II-related drug resistance. Biochem. Pharmacol. 45: 2025-2035, 1993.
Jensen P B, Sorensen B S, Sehested M, Grue P, Demant E J, and Hansen H H, Targeting the cytotoxicity of topoisomerase II-directed epipodophyllotoxins to tumor cells in acidic environments. Cancer Res. 54: 2959-2963, 1994.
Jensen P B, Sorensen B S, Sehested M, Grue P, Demant E J, and Hansen H H, Targeting the cytotoxicity of topoisomerase II-directed epipodophyllotoxins to tumor cells in acidic environments. Cancer Res. 54: 2959-2963, 1994.
Kaufmann S H and Svingen P A, Immunoblot analysis and band depletion assays. Methods Mol. Biol. 94:253-68: 253-268, 1999.
Kleymann G, Novel agents and strategies to treat herpes simplex virus infections. Expert. Opin. Investig. Drugs 12: 165-183, 2003.
Kohn K W, Erickson L C, Ewig R A, and Friedman C A, Fractionation of DNA from mammalian cells by alkaline elution. Biochemistry 15: 4629-4637, 1976.
Krynetskaia N F, Cai X, Nitiss J L, Krynetski E Y, and Relling M V, Thioguanine substitution alters DNA cleavage mediated by topoisomerase II. FASEB J. 14: 2339-2344, 2000.
Langer S W, Schmidt G, Sorensen M, Sehested M, and Jensen P B, Inhibitors of topoisomerase II as pH-dependent modulators of etoposide-mediated cytotoxicity. Clin. Cancer Res. 5: 2899-2907, 1999.
Larsen A K, Escargueil A E, and Skladanowski A, Catalytic topoisomerase II inhibitors in cancer therapy. Pharmacol Ther 99: 167-181, 2003.
Larsen A K, Escargueil A E, and Skladanowski A, From DNA damage to G2 arrest: the many roles of topoisomerase II. Prog. Cell Cycle Res. 5:295-300: 295-300, 2003.
Li T K and Liu L F, Tumor cell death induced by topoisomerase-targeting drugs. Annu. Rev. Pharmacol. Toxicol. 41:53-77: 53-77, 2001.
Lindsley J E, Use of a real-time, coupled assay to measure the ATPase activity of DNA topoisomerase II. Methods Mol. Biol. 95: 57-64, 2001.
Mojena M, Bosca L, Rider M H, Rousseau G G, and Hue L, Inhibition of 6-phosphofructo-2-kinase activity by mercaptopurines. Biochem. Pharmacol. 43: 671-678, 1992.
Morris S K, Baird C L, and Lindsley J E, Steady-state and rapid kinetic analysis of topoisomerase II trapped as the closed-clamp intermediate by ICRF-193. J. Biol. Chem. 275: 2613-2618, 2000.
Nitiss J L, Pourquier P, and Pommier Y, Aclacinomycin A stabilizes topoisomerase I covalent complexes. Cancer Res. 57: 4564-4569, 1997.
Perrin D, van Hille B, Barret J M, Kruczynski A, Etievant C, Imbert T, and Hill B T, F 11782, a novel epipodophylloid non-intercalating dual catalytic inhibitor of topoisomerases I and II with an original mechanism of action. Biochem. Pharmacol. 59: 807-819, 2000.
Renodon-Corniere A, Jensen L H, Nitiss J L, Jensen P B, and Sehested M, Interaction of human DNA topoisomerase II alpha with DNA: quantification by surface plasmon resonance. Biochemistry 41: 13395-13402, 2002.
Roca J and Wang J C, DNA transport by a type II DNA topoisomerase: evidence in favor of a two-gate mechanism. Cell 77: 609-616, 1994.
Roca J, Ishida R, Berger J M, Andoh T, and Wang J C, Antitumor bisdioxopiperazines inhibit yeast DNA topoisomerase II by trapping the enzyme in the form of a closed protein clamp. Proc. Natl. Acad Sci U.S.A 91: 1781-1785, 1994.
Sehested M and Jensen P B, Mapping of DNA topoisomerase II poisons (etoposide, clerocidin) and catalytic inhibitors (aclarubicin, ICRF-187) to four distinct steps in the topoisomerase II catalytic cycle. Biochem Pharmacol 51: 879-886, 1996.
Sehested M, Jensen P B, Sorensen B S, Holm B, Friche E, and Demant E J, Antagonistic effect of the cardioprotector (+)-1,2-bis(3,5-dioxopiperazinyl-1-yl)propane (ICRF-187) on DNA breaks and cytotoxicity induced by the topoisomerase II directed drugs daunorubicin and etoposide (VP-16). Biochem. Pharmacol. 46: 389-393, 1993.
Sehested M, Wessel I, Jensen L H, Holm B, Oliveri R S, Kenwrick S, Creighton A M, Nitiss J L, and Jensen P B, Chinese hamster ovary cells resistant to the topoisomerase II catalytic inhibitor ICRF-159: a Tyr49Phe mutation confers high-level resistance to bisdioxopiperazines. Cancer Res 58: 1460-1468, 1998.
Shapiro T A, Klein V A, and Englund P T, Isolation of kinetoplast DNA. Methods Mol. Biol. 94:61-7: 61-67, 1999.
Sorensen M, Sehested M, and Jensen P B, pH-dependent regulation of camptothecin-induced cytotoxicity and cleavable complex formation by the antimalarial agent chloroquine. Biochem. Pharmacol. 54: 373-380, 1997.
Tanabe K, Ikegami Y, Ishida R, and Andoh T, Inhibition of topoisomerase II by antitumor agents bis(2,6-dioxopiperazine) derivatives. Cancer Res 51: 4903-4908, 1991.
Wang H, Mao Y, Zhou N, Hu T, Hsieh TS, and Liu L F, Atp-bound topoisomerase ii as a target for antitumor drugs. J. Biol. Chem. 276: 15990-15995, 2001.
Wang J C, Cellular roles of DNA topoisomerases: a molecular perspective. Nat. Rev. Mol Cell Biol. 3: 430-440, 2002.
Wasserman R A, Austin C A, Fisher L M, and Wang J C, Use of yeast in the study of anticancer drugs targeting DNA topoisomerases: expression of a functional recombinant human DNA topoisomerase II alpha in yeast. Cancer Res. 53: 3591-3596, 1993.
Wessel I, Jensen L H, Jensen P B, Falck J, Rose A, Roerth M, Nitiss J L, and Sehested M, Human small cell lung cancer NYH cells selected for resistance to the bisdioxopiperazine topoisomerase II catalytic inhibitor ICRF-187 demonstrate a functional R162Q mutation in the Walker A consensus ATP binding domain of the alpha isoform. Cancer Res 59: 3442-3450, 1999.
Wessel I, Jensen L H, Renodon-Corniere A, Sorensen T K, Nitiss J L, Jensen P B, and Sehested M, Human small cell lung cancer NYH cells resistant to the bisdioxopiperazine ICRF-187 exhibit a functional dominant Tyr165Ser mutation in the Walker A ATP binding site of topoisomerase II alpha. FEBS Lett. 520: 161-166, 2002.
Wilstermann A M and Osheroff N, Stabilization of eukaryotic topoisomerase II-DNA cleavage complexes. Curr. Top. Med. Chem. 3: 321-338, 2003.
Yang X, Li W, Prescott E D, Burden S J, and Wang J C, DNA topoisomerase II beta and neural development. Science 287: 131-134, 2000.

1. A compound selected from compounds of the following formulae, and pharmaceutically acceptable salts, solvates, amides, esters, ethers, N-oxides, chemically protected forms, and prodrugs thereof, for use in a method of treatment or therapy of the human or animal body: 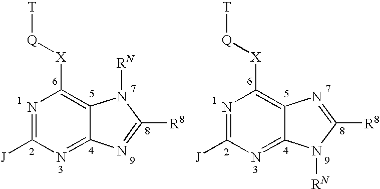
wherein:
J is independently:
—H, or
NR^{N1}R^{N2}.
X is independently:
—O—, or
—S—;
Q is independently:
a covalent bond,
C₁₋₇alkylene,
C₂₋₇alkenylene,
C₂₋₇alkynylene,
C₃₋₇cycloalkylene,
C₃₋₇cycloalkenylene, or
C₃₋₇cycloalkynylene;
T is independently:
a group A¹, or
a group A²;
A¹ is independently:
C₆₋₁₄carboaryl,
C₅₋₁₄heteroaryl,
C₃₋₁₂carbocyclic, or
C₃₋₁₂heterocyclic;
and is independently unsubstituted or substituted;
A² is independently:
—H,
—CN,
—OH, or
—O(C═O)—C₁₋₇alkyl;
R^{N} is independently —H or a nitrogen ring substituent;
R⁸ is independently —H or a ring substituent;
either: each of R^{N1} and R^{N2} is independently —H or a nitrogen substituent;
or: R^{N1} and R^{N2} taken together with the nitrogen atom to which they are attached form a ring having from 3 to 7 ring atoms.

2. A compound according to claim 1, wherein X is independently —O—.

3. A compound according to claim 1, wherein X is independently —S—.

4. A compound according to claim 1, wherein Q is independently a covalent bond.

5. A compound according to claim 1, wherein Q is independently C₁₋₇alkylene, C₂₋₇alkenylene, C₂₋₇alkynylene, C₃₋₇cycloalkylene, C₃₋₇cycloalkenylene, or C₃₋₇cycloalkynylene.

6. A compound according to claim 1, wherein Q is independently C₁₋₇alkylene, C₂₋₇alkenylene, or C₂₋₇alkynylene.

7. A compound according to claim 1, wherein Q is independently C₁₋₄alkylene, C₂₋₄alkenylene, or C₂₋₄alkynylene.

8. A compound according to claim 1, wherein Q is independently C₁₋₃alkylene, C₂₋₃alkenylene, or C₂₋₃alkynylene.

9. A compound according to claim 1, wherein Q is independently selected from —(CH₂)ₙ— where n is an integer from 1 to 7.

10. A compound according to claim 1, wherein Q is independently selected from —(CH₂)ₙ— where n is an integer from 1 to 4.

11. A compound according to claim 1, wherein Q is independently selected from —(CH₂)ₙ— where n is an integer from 1 to 3.

12. A compound according to claim 1, wherein Q is independently selected from —CH₂—, —CH₂CH₂—, —CH₂CH₂CH₂—, and —CH₂CH═CH—.

13. A compound according to claim 1, wherein J is —NR^{N1}R^{N2}.

14. A compound according to claim 1, wherein each of R^{N1} and R^{N2} is independently —H or a nitrogen substituent selected from:
C₁₋₇alkyl;
C₂₋₇alkenyl;
C₂₋₇alkynyl;
C₃₋₇cycloalkyl;
C₃₋₇cycloalkenyl;
C₃₋₇cycloalkynyl;
C₆₋₂₀carboaryl;
C₅₋₂₀heteroaryl;
C₃₋₂₀heterocyclyl;
C₆₋₂₀carboaryl-C₁₋₇alkyl;
C₅₋₂₀heteroaryl-C₁₋₇alkyl;
C₃₋₂₀heterocyclyl-C₁₋₇alkyl;
and is independently unsubstituted or substituted.

15. A compound according to claim 1, wherein each of R^{N1} and R^{N2} is independently —H or C₁₋₇alkyl, and is independently unsubstituted or substituted.

16. A compound according to claim 1, wherein each of R^{N1} and R^{N2} is independently —H or unsubstituted C₁₋₇alkyl.

17. A compound according to claim 1, wherein each of R^{N1} and R^{N2} is independently —H, -Me, or -Et.

18. A compound according to claim 1, wherein exactly one of R^{N1} and R^{N2} is —H, and the other is a nitrogen substituent.

19. A compound according to claim 1, wherein neither R^{N1} nor R^{N2} is —H.

20. A compound according to claim 1, wherein each of R^{N1} and R^{N2} is —H.

21. A compound according to claim 1, wherein the group —NR^{N1}R^{N2} is independently selected from:
—NH₂, —NHMe, —NHEt, —NH(nPr), —NH(iPr), —NH(nBu), —NH(iBu), —NH(sBu), —NH(tBu), —N(Me)₂, —N(Et)₂, —N(nPr)₂, —N(iPr)₂, —N(nBu)₂, —N(iBu)₂, —N(sBu)₂, —N(tBu)₂, —NH(Ph), —N(Ph)₂, —NH(CH₂Ph), —N(CH₂Ph)₂.

22. A compound according to claim 1, wherein the group —NR^{N1}R^{N2} is independently selected from: —NH₂, —NHMe, —NHEt, —N(Me)₂, —N(Et)₂.

23. A compound according to claim 1, wherein the group —NR^{N1}R^{N2} is independently —NH₂.

24. A compound according to claim 1, wherein R^{N1} and R^{N2} taken together with the nitrogen atom to which they are attached form a ring having from 3 to 7 ring atoms.

25. A compound according to claim 1, wherein R^{N1} and R^{N2} taken together with the nitrogen atom to which they are attached form a ring having from 5 to 7 ring atoms.

26. A compound according to claim 1, wherein the group —NR^{N1}R^{N2} is independently selected from:
aziridino;
azetidino;
pyrrolidin-N-yl, pyrrolin-N-yl, pyrrol-N-yl;
imidazolidin-N-yl, imidazolin-N-yl, imidazol-N-yl;
pyrazolidin-N-yl, pyrazolin-N-yl, pyrazol-N-yl;
piperidine-N-yl, piperazin-N-yl, pyridin-N-yl;
morpholino; and
azepin-N-yl.

27. A compound according to claim 1, wherein J is independently —H.

28. A compound according to claim 1, wherein R^{N} is independently —H or a nitrogen ring substituent selected from:
C₁₋₇alkyl;
C₂₋₇alkenyl;
C₂₋₇alkynyl;
C₃₋₇cycloalkyl;
C₃₋₇cycloalkenyl;
C₃₋₇cycloalkynyl;
C₆₋₂₀carboaryl;
C₅₋₂₀heteroaryl;
C₃₋₂₀heterocyclyl;
C₆₋₂₀carboaryl-C₁₋₇alkyl;
C₅₋₂₀heteroaryl-C₁₋₇alkyl;
C₃₋₂₀heterocyclyl-C₁₋₇alkyl;
and is independently unsubstituted or substituted.

29. A compound according to claim 1, wherein R^{N} is independently —H or C₁₋₇alkyl, and is independently unsubstituted or substituted.

30. A compound according to claim 1, wherein R^{N} is independently —H or unsubstituted C₁₋₇alkyl.

31. A compound according to claim 1, wherein R^{N} is independently —H, -Me, or -Et.

32. A compound according to claim 1, wherein R^{N} is independently —H.

33. A compound according to claim 1, wherein R^{N} is independently —H or tetrahydrofuranyl, and is independently unsubstituted or substituted.

34. A compound according to claim 1, wherein R^{N} is independently —H or morpholino-methyl, piperidino-methyl, or piperazino-methyl, and is independently unsubstituted or substituted.

35. A compound according to claim 1, wherein R^{N} is independently selected from:

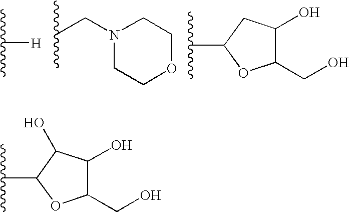

36. A compound according to claim 1, wherein T is independently A¹.

37. A compound according to claim 1, wherein A¹ is independently:
C₆₋₁₄carboaryl, or
C₅₋₁₄heteroaryl;
and is independently unsubstituted or substituted.

38. A compound according to claim 1, wherein A¹ is independently:
C₆₋₁₂carboaryl, or
C₅₋₁₂heteroaryl;
and is independently unsubstituted or substituted.

39. A compound according to claim 1, wherein A¹ is independently:
C₆₋₁₀carboaryl, or
C₅₋₁₀heteroaryl;
and is independently unsubstituted or substituted.

40. A compound according to claim 1, wherein A¹ is independently:
monocyclic or bicyclic C₆₋₁₀carboaryl, or
monocyclic or bicyclic C₅₋₁₀heteroaryl;
and is independently unsubstituted or substituted.

41. A compound according to claim 1, wherein A¹ is independently:
monocyclic C₆carboaryl, or
monocyclic C₅₋₆heteroaryl;
and is independently unsubstituted or substituted.

42. A compound according to claim 1, wherein A¹ is independently: phenyl, naphthyl, pyridyl, pyrimidyl, pyrrolyl, imidazolyl, furanyl, thienyl, thiazoyl, or benzofurazanyl; and is independently unsubstituted or substituted.

43. A compound according to claim 1, wherein A¹ is independently: phenyl, naphthyl, pyrididyl, pyrrolyl, furanyl, thienyl, and thiazolyl; and is independently unsubstituted or substituted.

44. A compound according to claim 1, wherein A¹ is independently: phenyl, pyrimidyl, imidazolyl, or benzofurazanyl; and is independently unsubstituted or substituted.

45. A compound according to claim 1, wherein A¹ is independently phenyl; and is independently unsubstituted or substituted.

46. A compound according to claim 1, wherein A¹ is independently pyrimidyl; and is independently unsubstituted or substituted.

47. A compound according to claim 1, wherein A¹ is independently imidazolyl; and is independently unsubstituted or substituted.

48. A compound according to claim 1, wherein A¹ is independently benzofurazanyl; and is independently unsubstituted or substituted.

49. A compound according to claim 1, wherein A¹ is independently:
C₃₋₁₂carbocyclic, or
C₃₋₁₂heterocyclic;
and is independently unsubstituted or substituted.

50. A compound according to claim 1, wherein A¹ is independently:
C₅₋₁₀carbocyclic, or
C₅₋₁₀heterocyclic;
and is independently unsubstituted or substituted.

51. A compound according to claim 1, wherein A¹ is independently:
monocyclic or bicyclic C₃₋₁₂carbocyclic, or
monocyclic or bicyclic C₃₋₁₂heterocyclic;
and is independently unsubstituted or substituted.

52. A compound according to claim 1, wherein A¹ is independently:
C₅₋₈carbocyclic, or
C₅₋₈heterocyclic;
and is independently unsubstituted or substituted.

53. A compound according to claim 1, wherein A¹ is independently:
monocyclic C₅₋₈carbocyclic, or
monocyclic C₅₋₈heterocyclic;
and is independently unsubstituted or substituted.

54. A compound according to claim 1, wherein A¹ is independently: cyclopentyl, cyclohexyl, tetrahydrofuranyl, tetrahydropyranyl, dioxanyl, pyrrolidinyl, piperidinyl, or piperzinyl; and is independently unsubstituted or substituted.

55. A compound according to claim 1, wherein A¹ is independently cyclohexyl; and is independently unsubstituted or substituted.

56. A compound according to claim 1, wherein substituents on the cyclic group A¹, if present, are independently selected from:
(1) carboxylic acid; (2) ester; (3) amido or thioamido; (4) acyl; (5) halo; (6) cyano; (7) nitro; (8) hydroxy; (9) ether; (10) thiol; (11) thioether; (12) acyloxy; (13) carbamate; (14) amino; (15) acylamino orthioacylamino; (16) aminoacylamino or aminothioacylamino; (17) sulfonamino; (18) sulfonyl; (19) sulfonate; (20) sulfonamido; (21) oxo; (22) imino; (23) hydroxyimino; (24) C₅₋₂₀aryl-C₁₋₇alkyl; (25) C₅₋₂₀aryl; (26) C₃₋₂₀heterocyclyl; (27) C₁₋₇alkyl; (28) bi-dentate di-oxy groups.

57. A compound according to claim 1, wherein substituents on the cyclic group A¹, if present, are independently selected from:
(1) —C(═O)OH;
(2) —C(═O)OR¹, wherein R¹ is independently as defined in (24), (25), (26) or (27);
(3) —C(═O)NR²R³ or —C(═S)NR²R³, wherein each of R² and R³ is independently —H; or as defined in (24), (25), (26) or (27); or R² and R³ taken together with the nitrogen atom to which they are attached form a ring having from 3 to 7 ring atoms;
(4) —C(═O)R⁴, wherein R⁴ is independently —H, or as defined in (24), (25), (26) or (27);
(5) —F, —Cl, —Br, —I;
(6) —CN;
(7) —NO₂;
(8) —OH;
(9) —OR⁵, wherein R⁵ is independently as defined in (24), (25), (26) or (27);
(10) —SH;
(11) —SR⁶, wherein R⁶ is independently as defined in (24), (25), (26) or (27);
(12) —OC(═O)R⁷, wherein R⁷ is independently as defined in (24), (25), (26) or (27);
(13) —OC(═O)NR⁸R⁹, wherein each of R⁸ and R⁹ is independently —H; or as defined in (24), (25), (26) or (27); or R⁸ and R⁹ taken together with the nitrogen atom to which they are attached form a ring having from 3 to 7 ring atoms;
(14) —NR¹⁰R¹¹, wherein each of R¹⁰ and R¹¹ is independently —H; or as defined in (24), (25), (26) or (27); or R¹⁰ and R¹¹ taken together with the nitrogen atom to which they are attached form a ring having from 3 to 7 ring atoms;
(15) —NR¹²C(═O)R¹³ or —NR¹²C(═S)R¹³, wherein R¹² is independently —H; or as defined in (24), (25), (26) or (27); and R¹³ is independently —H, or as defined in (24), (25), (26) or (27);
(16) —NR¹⁴C(═O)NR¹⁵R¹⁶ or —NR¹⁴C(═S)NR¹⁵R¹⁶, wherein R¹⁴ is independently —H; or as defined in (24), (25), (26) or (27); and each of R¹⁵ and R¹⁶ is independently —H; or as defined in (24), (25), (26) or (27); or R¹⁵ and R¹⁶ taken together with the nitrogen atom to which they are attached form a ring having from 3 to 7 ring atoms;
(17) —NR¹⁷SO₂R¹⁸, wherein R¹⁷ is independently —H; or as defined in (24), (25), (26) or (27); and R¹⁸ is independently —H, or as defined in (24), (25), (26) or (27);
(18) —SO₂R¹⁹, wherein R¹⁹ is independently as defined in (24), (25), (26) or (27);
(19) —OSO₂R²⁰ and wherein R²⁰ is independently as defined in (24), (25), (26) or (27);
(20) —SO₂NR²¹R²², wherein each of R²¹ and R²² is independently —H; or as defined in (24), (25), (26) or (27); or R²¹ and R²² taken together with the nitrogen atom to which they are attached form a ring having from 3 to 7 ring atoms;
(21) ═O;
(22) ═NR²³, wherein R²³ is independently —H; or as defined in (24), (25), (26) or (27);
(23) ═NOR²⁴, wherein R²⁴ is independently —H; or as defined in (24), (25), (26) or (27);
(24) C₅₋₂₀aryl-C₁₋₇alkyl, for example, wherein C₅₋₂₀aryl is as defined in (25); unsubstituted or substituted, e.g., with one or more groups as defined in (1) to (28);
(25) C₅₋₂₀aryl, including C₆₋₂₀carboaryl and C₅₋₂₀heteroaryl; unsubstituted or substituted, e.g., with one or more groups as defined in (1) to (28);
(26) C₃₋₂₀heterocyclyl; unsubstituted or substituted, e.g., with one or more groups as defined in (1) to (28);
(27) C₁₋₇alkyl, C₂₋₇alkenyl, C₂₋₇alkynyl, C₃₋₇cycloalkyl, C₃₋₇cycloalkenyl, C₃₋₇cycloalkynyl, unsubstituted or substituted, e.g., with one or more groups as defined in (1) to (26) and
(28) —O—R²⁵—O—, wherein R²⁵ is independently saturated C₁₋₃alkyl, and is independently unsubstituted or substituted with one or more (e.g., 1, 2, 3, 4) substituents as defined in (5).

58. A compound according to claim 57, wherein (27) C₁₋₇alkyl, unsubstituted or substituted is:
Unsubstituted C₁₋₇alkyl;
halo-C₁₋₇alkyl;
amino-C₁₋₇alkyl;
amido-C₁₋₇alkyl;
acylamido-C₁₋₇alkyl;
carboxy-C₁₋₇alkyl;
acyl-C₁₋₇alkyl;
hydroxy-C₁₋₇alkyl; and
C₁₋₇alkoxy-C₁₋₇alkyl.

59. A compound according to claim 1, wherein substituents on the cyclic group A¹, if present, are independently selected from:
(1) —C(═O)OH;
(2) —C(═O)OMe, —C(═O)OEt, —C(═O)O(iPr), —C(═O)O(tBu); —C(═O)O(cPr); —C(═O)OCH₂CH₂OH, —C(═O)OCH₂CH₂OMe, —C(═O)OCH₂CH₂OEt; —C(═O)OPh, —C(═O)OCH₂Ph;
(3) —(C═O)NH₂, —(C═O)NMe₂, —(C═O)NEt₂, —(C═O)N(iPr)₂, —(C═O)N(CH₂CH₂OH)₂; —(C═O)-morpholino, —(C═O)NHPh, —(C═O)NHCH₂Ph;
(4) —C(═O)H, —(C═O)Me, —(C═O)Et, —(C═O)(tBu), —(C═O)-cHex, —(C═O)Ph; —(C═O)CH₂Ph;
(5) —F, —Cl, —Br, —I;
(6) —CN;
(7) —NO₂;
(8) —OH;
(9) —OMe, —OEt, —O(iPr), —O(tBu), —OPh, —OCH₂Ph; —OCF₃, —OCH₂CF₃; —OCH₂CH₂OH, —OCH₂CH₂OMe, —OCH₂CH₂OEt; —OCH₂CH₂NH₂, —OCH₂CH₂NMe₂, —OCH₂CH₂N(iPr)₂; —OPh-Me, —OPh-OH, —OPh-OMe, —OPh-F, —OPh-Cl, —OPh-Br, —OPh-I;
(10) —SH;
(11) —SMe, —SEt, —SPh, —SCH₂Ph;
(12) —OC(═O)Me, —OC(═O)Et, —OC(═O)(iPr), —OC(═O)(tBu); —OC(═O)(cPr); —OC(═O)CH₂CH₂OH, —OC(═O)CH₂CH₂OMe, —OC(═O)CH₂CH₂OEt; —OC(═O)Ph, —OC(═O)CH₂Ph;
(13) —OC(═O)NH₂, —OC(═O)NHMe, —OC(═O)NMe₂, —OC(═O)NHEt, —OC(═O)NEt₂, —OC(═O)NHPh, —OC(═O)NCH₂Ph;
(14) —NH₂, —NHMe, —NHEt, —NH(iPr), —NMe₂, —NEt₂, —N(iPr)₂, —N(CH₂CH₂OH)₂; —NHPh, —NHCH₂Ph; piperidino, piperazino, morpholino;
(15) —NH(C═O)Me, —NH(C═O)Et, —NH(C═O)ₙPr, —NH(C═O)Ph, —NHC(═O)CH₂Ph; —NMe(C═O)Me, —NMe(C═O)Et, —NMe(C═O)Ph, —NMeC(═O)CH₂Ph;
(16) —NH(C═O)NH₂, —NH(C═O)NHMe, —NH(C═O)NHEt, —NH(C═O)NPh, —NH(C═O)NHCH₂Ph; —NH(C═S)NH₂, —NH(C═S)NHMe, —NH(C═S)NHEt, —NH(C═S)NPh, —NH(C═S)NHCH₂Ph;
(17) —NHSO₂Me, —NHSO₂Et, —NHSO₂Ph, —NHSO₂PhMe, —NHSO₂CH₂Ph; —NMeSO₂Me, —NMeSO₂Et, —NMeSO₂Ph, —NMeSO₂PhMe, —NMeSO₂CH₂Ph;
(18) —SO₂Me, —SO₂CF₃, —SO₂Et, —SO₂Ph, —SO₂PhMe, —SO₂CH₂Ph;
(19) —OSO₂Me, —OSO₂CF₃, —OSO₂Et, —OSO₂Ph, —OSO₂PhMe, —OSO₂CH₂Ph;
(20) —SO₂NH₂, —SO₂NHMe, —SO₂NHEt, —SO₂NMe₂, —SO₂NEt₂, —SO₂-morpholino, —SO₂NHPh, —SO₂NHCH₂Ph;
(21) ═O;
(22) ═NH, ═NMe; ═NEt;
(23) ═NOH, ═NOMe, ═NOEt, ═NO(nPr), ═NO(iPr), ═NO(cPr), ═NO(CH₂-cPr);
(24) —CH₂Ph, —CH₂Ph-Me, —CH₂Ph-OH, —CH₂Ph-F, —CH₂Ph-Cl;
(25) -Ph, -Ph-Me, -Ph-OH, -Ph-OMe, -Ph-NH₂, -Ph-F, -Ph-Cl, -Ph-Br, -Ph-I; pyridyl, pyrazinyl, pyrimidinyl, pyridazinyl, furanyl, thiophenyl, pyrrolyl, imidazolyl, pyrazolyl, oxazolyl, thiazolyl, thiadiazolyl;
(26) pyrrolidinyl, imidazolidinyl, pyrazolidinyl, piperidinyl, piperazinyl, azepinyl, tetrahydrofuranyl, tetrahydropyranyl, morpholinyl, azetidinyl;
(27) -Me, -Et, -nPr, -iPr, -nBu, -iBu, -sBu, -tBu, -nPe;
-cPr, -cHex; —CH═CH₂, —CH₂—CH═CH₂;
—CF₃, —CHF₂, —CH₂F, —CCl₃, —CBr₃, —CH₂CH₂F, —CH₂CHF₂, and —CH₂CF₃;
—CH₂OH, —CH₂OMe, —CH₂OEt, —CH₂NH₂, —CH₂NMe₂;
—CH₂CH₂OH, —CH₂CH₂OMe, —CH₂CH₂OEt, —CH₂CH₂CH₂NH₂, —CH₂CH₂NMe₂;
(28) —O—CH₂—O—, —O—CH₂—CH₂—O—, —O—CH₂—CH₂—CH₂—O—, —O—CF₂—O—, and —O—CF₂—CF₂—O—.

60. A compound according to claim 1, wherein substituents on the cyclic group A¹, if present, are independently selected from:
(2) —C(═O)OMe, —C(═O)OEt;
(5) —F, —Cl, —Br, —I;
(7) —NO₂;
(8) —OH;
(9) —OMe, —OEt;
(11) —SMe, —SEt;
(12) —OC(═O)Me, —OC(═O)Et;
(14) —NH₂, —NHMe, —NHEt, —NMe₂, —NEt₂;
(27) -Me, and -Et.

61. A compound according to claim 1, wherein T, is independently A².

62. A compound according to claim 61, wherein A² is independently:
—H;
—CN;
—OH; or
—O(C═O)—C₁₋₇alkyl.

63. A compound according to claim 61, wherein A², is independently:
—H;
—CN;
—OH; or
—O(C═O)—C₁₋₇alkyl;
with the proviso that Q is not a covalent bond.

64. A compound according to claim 61, wherein A² is independently —H, with the proviso that Q is not a covalent bond.

65. A compound according to claim 61, wherein A² is independently —CN, with the proviso that Q is not a covalent bond.

66. A compound according to claim 61, wherein A² is independently —OH or —O(C═O)—C₁₋₇alkyl, with the proviso that Q is not a covalent bond.

67. A compound according to claim 61, wherein A² is independently —OH or —O(C═O)Me, with the proviso that Q is not a covalent bond.

68. A compound according to claim 1, wherein R⁸ is independently —H or a monovalent monodentate substituent selected from those defined for (1) through (20) and (24) through (27) in any one of claims 56 to 60.

69. A compound according to claim 1, wherein R⁸ is independently —H.

70. A compound according to claim 1, selected from the following compounds, and pharmaceutically acceptable salts, solvates, amides, esters, ethers, N-oxides, chemically protected forms, and prodrugs thereof:

| | |
|---|---|
| 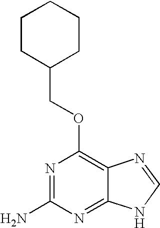 | NU2058 |
| 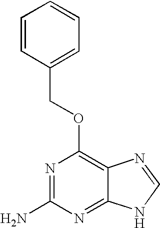 | O⁶-benzylguanine |
| 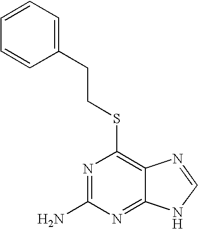 | NSC35866 |
| 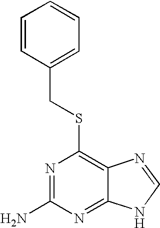 | NSC15747 |
| 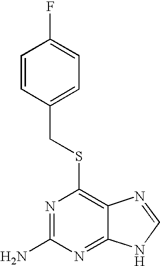 | NSC35865 |
| 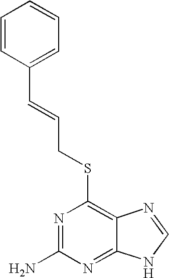 | NSC36824 |
| 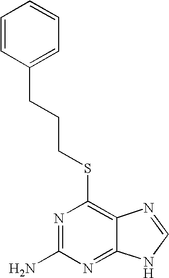 | NSC39328 |
| 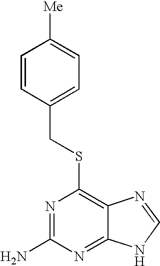 | NSC46384 |

71. A compound according to claim 1, selected from the following compounds, and pharmaceutically acceptable salts, solvates, amides, esters, ethers, N-oxides, chemically protected forms, and prodrugs thereof:

| | |
|---|---|
| 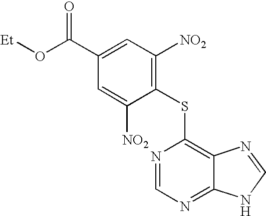 | NSC244708 |
| 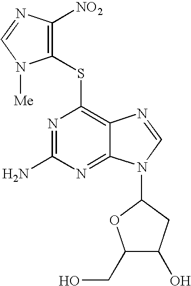 | NSC172614 |
| 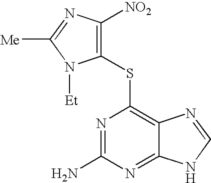 | NSC42375 |
| 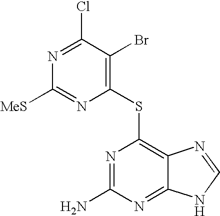 | NSC52383 |
| 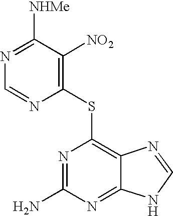 | NSC38732 |
| 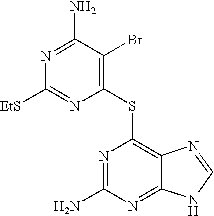 | NSC52388 |
| 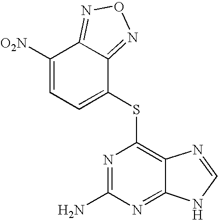 | NSC348401 |
| 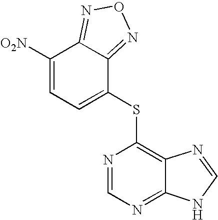 | NSC348402 |
| 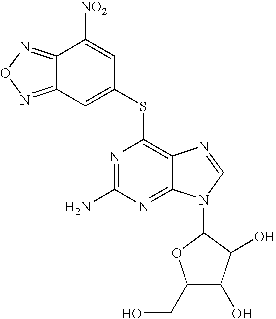 | NSC348400 |

72. A compound according to claim 1, selected from the following compounds, and pharmaceutically acceptable salts, solvates, amides, esters, ethers, N-oxides, chemically protected forms, and prodrugs thereof:

| | |
|---|---|
| 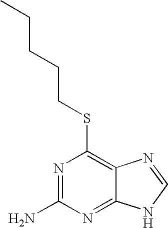 | NSC35862 |
| 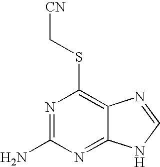 | NSC39331 |
| 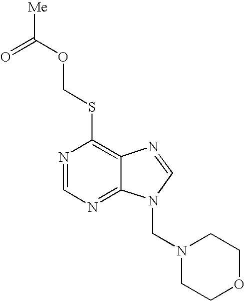 | NSC647471 |

73. A compound as defined in claim 1 for use in combination with a topoisomerase II poison in a method of treatment of the human or animal body by therapy.

74. A compound according to claim 73, wherein the topoisomerase II poison is an anthracycline or an epipodophyllotoxin.

75. A compound according to claim 73, wherein the topoisomerase II poison is an anthracycline selected from: doxorubicin, idarubicin, epirubicin, aclarubicin, mitoxantrone, dactinomycin, bleomycin, mitomycin, carubicin, pirarubicin, daunorubicin, daunomycin, 4-iodo-4-deoxy-doxorubicin, N,N-dibenzyl-daunomycin, morpholinodoxorubicin, aclacinomycin, duborimycin, menogaril, nogalamycin, zorubicin, marcellomycin, detorubicin, annamycin, 7-cyanoquinocarcinol, deoxydoxorubicin, valrubicin, GPX-100, MEN-10755, and KRN5500.

76. A compound according to claim 73, wherein the topoisomerase II poison is an epipodophyllotoxin selected from: etoposide, etoposide phosphate, teniposide, tafluposide, VP-16213, and NK-611.

77. A compound according to claim 73, wherein the topoisomerase II poison is etoposide.

78. Use of a compound as defined in claim 1 in the manufacture of a medicament for use in the treatment of a disease or condition that is ameliorated by the catalytic inhibition of topoisomerase II.

79. Use according to claim 78, wherein the treatment is prevention or treatment of tissue damage associated with extravasation of a topoisomerase II poison.

80. Use according to claim 78, wherein the treatment is prevention or treatment of tissue damage associated with extravasation of a topoisomerase II poison in a patient receiving treatment with said topoisomerase II poison.

81. Use according to claim 79, wherein the medicament is for systemic administration.

82. Use according to claim 79, wherein the medicament is for local administration.

83. Use according to claim 79, wherein the topoisomerase II poison is an anthracycline or an epipodophyllotoxin.

84. Use according to claim 79, wherein the topoisomerase II poison is an anthracycline selected from: doxorubicin, idarubicin, epirubicin, aclarubicin, mitoxantrone, dactinomycin, bleomycin, mitomycin, carubicin, pirarubicin, daunorubicin, daunomycin, 4-iodo-4-deoxy-doxorubicin, N,N-dibenzyl-daunomycin, morpholinodoxorubicin, aclacinomycin, duborimycin, menogaril, nogalamycin, zorubicin, marcellomycin, detorubicin, annamycin, 7-cyanoquinocarcinol, deoxydoxorubicin, valrubicin, GPX-100, MEN-10755, and KRN5500.

85. Use according to claim 79, wherein the topoisomerase II poison is an epipodophyllotoxin selected from: etoposide, etoposide phosphate, teniposide, tafluposide, VP-16213, and NK-611.

86. Use according to claim 79, wherein the topoisomerase II poison is etoposide.

87. Use of a compound as defined in claim 1 in the manufacture of a medicament for use in combination with a topoisomerase II poison, in the treatment of a disease or condition that is ameliorated by the catalytic inhibition of topoisomerase II.

88. Use according to claim 87, wherein the treatment is treatment of a proliferative condition.

89. Use according to claim 87, wherein the treatment is treatment of cancer.

90. Use according to claim 87, wherein the treatment is treatment of solid tumour cancer.

91. Use according to claim 87, wherein the treatment is treatment of a proliferative condition of the central nervous system (CNS).

92. Use according to claim 87, wherein the treatment is treatment of a tumour of the central nervous system (CNS).

93. Use according to claim 87, wherein the treatment is treatment of brain cancer.

94. Use according to claim 87, wherein the topoisomerase II poison is an anthracycline or an epipodophyllotoxin.

95. Use according to claim 87, wherein the topoisomerase II poison is an anthracycline selected from: doxorubicin, idarubicin, epirubicin, aclarubicin, mitoxantrone, dactinomycin, bleomycin, mitomycin, carubicin, pirarubicin, daunorubicin, daunomycin, 4-iodo-4-deoxy-doxorubicin, N,N-dibenzyl-daunomycin, morpholinodoxorubicin, aclacinomycin, duborimycin, menogaril, nogalamycin, zorubicin, marcellomycin, detorubicin, annamycin, 7-cyanoquinocarcinol, deoxydoxorubicin, valrubicin, GPX-100, MEN-10755, and KRN5500.

96. Use according to claim 87, wherein the topoisomerase II poison is an epipodophyllotoxin selected from: etoposide, etoposide phosphate, teniposide, tafluposide, VP-16213, and NK-611.

97. Use according to claim 87, wherein the topoisomerase II poison is etoposide.

98. A method of inhibiting topoisomerase II in a cell, in vitro or in vivo, comprising contacting the cell with an effective amount of a compound as defined in claim 1.

99. A method of treatment comprising administering to a patient in need of treatment a therapeutically effective amount of a compound as defined in claim 1.

100. A method of treatment comprising administering to a patient in need of treatment a therapeutically effective amount of a compound as defined in claim 1 and a topoisomerase II poison.

101. A method of targeting the cytotoxicity of a topoisomerase II poison, comprising administering a compound as defined in claim 1, in combination with said topoisomerase II poison.

102. A method according to claim 101, wherein the targeting is targeting to a solid tumour.

103. A method according to claim 101, wherein the targeting is targeting to the central nervous systems (CNS).

104. A method of permitting increased dosage of a topoisomerase II poison in therapy, comprising administering a compound as defined in claim 1, in combination with said topoisomerase II poison.


SEQUENCE LISTING (2 sequences)

SEQ ID NO: 1 - DNA, 20 bp, Homo sapiens
gaaatacgagactgctcggc

SEQ ID NO: 2 - DNA, 23 bp, Homo sapiens
ttaaaactcatagtcttcatcag